(19) United States
(12) Patent Application Publication
(10) Pub. No.: US 2012/0294889 A1
(43) Pub. Date: Nov. 22, 2012

(54) Chimeric Flavivirus Vaccines

(75) Inventors: Thomas P. Monath, Townsend, MA (US); Nikolaos Vasilakis, Galveston, TX (US)

(73) Assignee: PAXVAX, INC., Menlo Park, CA (US)

(21) Appl. No.: 13/294,954

(22) Filed: Nov. 11, 2011

Related U.S. Application Data
(60) Provisional application No. 61/412,829, filed on Nov. 12, 2010.
(60) Provisional application No. 61/439,225, filed on Feb. 3, 2011.
(60) Provisional application No. 61/499,591, filed on Jun. 21, 2011.
(60) Provisional application No. 61/522,295, filed on Aug. 11, 2011.

(51) Int. Cl. C12N 15/86; C12N 7/01; C12N 7/04; C12N 7/06; A61K 39/295; A61P 31/14

(52) U.S. Cl. 424/199.1; 424/202.1; 435/320.1; 435/236; 435/238; 435/235.1

(57) ABSTRACT

The invention provides chimeric flavivirus vectors encoding one or more structural proteins from a first flavivirus with a low level of replication in a cell, such as dengue virus and yellow fever virus, and a backbone from a second flavivirus with a high level of replication in the cell, such as the Rio Bravo virus or the Uganda S virus. The chimeric flaviviruses encoded by the chimeric flavivirus vectors of the invention can be used to vaccinate subjects to prevent infection from infectious flaviviruses, including dengue viruses and yellow fever viruses.

### CROSS REFERENCE TO RELATED APPLICATIONS

This application claims priority to U.S. Provisional Application Ser. No. 61/412,829 filed Nov. 12, 2010; U.S. Provisional Application Ser. No. 61/439,225, filed Feb. 3, 2011; U.S. Provisional Application Ser. No. 61/499,591, filed Jun. 21, 2011; and U.S. Provisional Application Ser. No. 61/522,295, filed Aug. 11, 2011, each of which is herein incorporated by reference in its entirety for all purposes.

### DESCRIPTION OF THE TEXT FILE SUBMITTED ELECTRONICALLY

The contents of the text file submitted electronically herewith are incorporated herein by reference in their entirety: A computer readable format copy of the Sequence Listing (filename: PAXV_{—}013_{—}04US_SeqList_ST25.txt, date recorded: Nov. 10, 2011, file size: 9 kilobytes).

### BACKGROUND OF THE INVENTION

Flaviviruses are members of the genus Flavivirus, which is classified within the family Flaviviridae. Many flaviviruses are significantly pathogenic for humans and other mammals. Two of the most important pathogens of the Flavivirus genus are dengue (serotypes 1, 2, 3 and 4) and yellow fever.

Dengue viruses are mosquito-borne viruses and are small (˜50 nm), spherical viruses comprising an envelope composed of lipid and two viral proteins (the membrane and envelope proteins) that package and protect the positive-sense single-strand RNA genome and capsid protein. The ˜11 kilobase RNA genome contains a single long open reading frame encoding three structural proteins (the capsid protein, pre-membrane and envelope) and 7 non-structural proteins that are needed for replication but which remain in the infected cell and are not incorporated into the mature virus particle. The pre-membrane protein is truncated during virus assembly to leave the mature membrane protein incorporated into the viral envelope. The non-structural genes encode enzymes, including protease involved in post-translational processing of viral proteins, and helicase and polymerase involved in replication of the viral RNA. At both the 3′ and 5′ termini of the viral genome there are short non-coding regions which form structural elements that allow for the polymerase to anchor and initiate replication of the positive- and negative-polarity RNA strands during replication. The dengue virus genome organization and function of the encoded viral proteins are reviewed in Lindenbach et al., 2003, Adv Virus Res 59: 23-61, and Perera et al., 2010, Curr Opin Microbiol 11: 369-77.

Dengue is endemic and epidemic in tropical regions of the world, with up to 100 million persons infected every year. (Gubler D J, 2004, Comp Immunol Microbiol Infect Dis. 27: 319-30; Halstead S B, 2007, Lancet 370: 1644-52). Humans infected with dengue viruses experience febrile illness with severe muscle pains, headache and rash (dengue fever). Severe dengue, also known as dengue hemorrhagic fever/dengue shock syndrome (DHF/DSS), is an immunopathological disease that occurs in individuals, mainly children, who sustain sequential infections with different dengue virus serotypes. Therefore, immunity to all four dengue serotypes should be evoked simultaneously by a successful vaccine, as described in detail below.

Like the dengue virus, the yellow fever virus is in the Flavivirus genus, in the family Flaviviridae. The yellow fever virus is maintained in nature in a transmission cycle involving nonhuman primates and mosquitoes in certain tropical areas of Africa and the Americas. Yellow fever occurs in sporadic human cases and periodically amplifies into epidemics. Other parts of the world, including coastal regions of South America, the Caribbean Islands, and Central and North America, are infested with the mosquito vector capable of transmitting the virus and are therefore considered at risk for introduction and spread of yellow fever epidemics. Illness from the yellow fever virus ranges in severity from a self-limited febrile illness to severe hepatitis and fatal hemorrhagic disease. Unvaccinated humans, including both residents of and travelers to yellow fever endemic areas are at significant risk of yellow fever infection when occupational and other activities bring them in contact with infected mosquitoes. There is no specific treatment for yellow fever. Steps to prevent yellow fever include the use of insect repellent, protective clothing, and vaccination with the available, but risky attenuated vaccine.

Live, attenuated vaccines for the dengue virus and the yellow fever virus are available and/or in clinical development. There are problems, however, associated with the use of these live, attenuated vaccines. For yellow fever, live, attenuated vaccines have been produced from the 17D substrain, but adverse events associated with the attenuated vaccine can lead to a severe infection with the live 17D virus, and serious and fatal adverse neurotropic and viscerotropic events, the latter resembling the severe infection caused by the wild-type yellow fever virus. For dengue virus, current investigational vaccines are a mixture of four live viruses (each representing one of the four dengue serotypes). Unfortunately, the use of four live viruses has caused interference between the four components, thus preventing successful immunization on the first dose. Indeed, these live dengue virus vaccines require multiple doses of the tetravalent mixture at intervals of 3-6 months to achieve immunity against all four serotypes. Thus, the dosing regimen can take as long as 12 months to complete. Moreover, usually only 25-50% of subjects given two doses of live vaccines develop neutralizing antibodies to all four serotypes. Thus, for both yellow fever and dengue, better methods of immunization are necessary.

A successful vaccine development strategy is to grow the virus in an acceptable cell culture system, to harvest the virus which is released into the cell culture medium, and to inactivate the virus or produce a virus incapable of replication in mammals, preserving its structure and epitopes. Non-replicating vaccine components in a tetravalent mixture would not interfere with one another, and it is expected that a balanced response to all four serotypes, and high titers of antibody could be evoked using this method.

Unfortunately, dengue viruses and yellow fever viruses grow to maximum levels of only about 10⁷ PFU/mL, 10 to 100-fold (or more) lower than other Flaviviruses against which successful and approved inactivated vaccines have been developed, such as Japanese encephalitis and tick-borne encephalitis. This severely restricts the ability to manufacture inactivated whole virion vaccines against dengue and yellow fever.

Therefore, methods for growing dengue virus and/or yellow fever virus at high yields (greater than or equal to 10⁸ PFU/mL) for preparing whole virion vaccines would be highly desirable. The efficacy of such a dengue or yellow fever vaccine would be expected to be similar to inactivated vaccines against other Flaviviruses, such as Japanese encephalitis (IXIARO® Prescribing information, 2009, Intercell AG) and tick-borne encephalitis (Schöndorf et al., 2007, Vaccine 25: 1470-5). The present invention meets the need for highly-replicating dengue viruses and yellow fever viruses by providing chimeric viral vectors, viruses encoded thereof as well as related vaccines comprising dengue virus or yellow fever-virus components and methods for growing these viruses in cell cultures to levels that allow efficient manufacture of inactivated, whole virion vaccines.

### SUMMARY OF THE INVENTION

The present invention is based, in part, on the discovery that one or more structural proteins from a first flavivirus with low level of replication in cells can be integrated into a second flavivirus with a high level of replication in cells, e.g., one or more structural proteins of a first flavivirus can be used to replace one or more structural proteins of a second flavivirus. Accordingly, the present invention provides chimeric flavivirus vectors, chimeric flaviviruses encoded thereof, and vaccines containing chimeric flavivirus vectors or chimeric flavivirus(es). In addition, the present invention also provides methods of vaccination or inducing an immune response using the vaccines of the present invention.

Accordingly, in one aspect, the invention provides a chimeric flavivirus vector encoding a structural protein from a first flavivirus with a low level of replication in a cell and a backbone from a second flavivirus with a high level of replication in the cell. In an alternative aspect, the invention provides a chimeric flavivirus vector encoding an envelope protein from a first flavivirus with a low level of replication in a cell and one or more non-structural proteins from a second flavivirus with a high level of replication in the cell.

In one embodiment, the structural protein is an envelope protein. In another embodiment, the structural protein comprises an envelope protein and a pre-membrane protein. In some embodiments, the backbone comprises a capsid protein, the non-structural proteins, and the 3′ and 5′ non-coding termini. In one embodiment, the first flavivirus with a low level of replication in a cell is selected from the group consisting of dengue virus and yellow fever virus. In another embodiment, the first flavivirus is a dengue virus selected from the group consisting of dengue type 1 (DEN1) virus, dengue type 2 (DEN2) virus, dengue type 3 (DEN3) virus, and dengue type 4 (DEN4) virus.

In certain embodiments, the second flavivirus with a high level of replication in the cell is a flavivirus selected from the Rio Bravo taxonomic group. For example, the flavivirus of the Rio Bravo taxonomic group may be selected from the group consisting of the Rio Bravo (sensu stricto) virus, the Montana Myotis Leukoencephalitis virus, the Dakar Bat virus, the Phnom Penh Bat virus, the Carey Island virus, and the Bukalasa Bat virus.

In certain other embodiments, the second flavivirus with a high level of replication in the cell is a flavivirus selected from the Uganda S taxonomic group. For example, the flavivirus of the Uganda S taxonomic group may be selected from the group consisting of the Uganda S virus, the Banzi virus, and the Jugra virus.

In still other embodiments, the second flavivirus with a high level of replication in the cell is a flavivirus selected from the mosquito-associated flaviviruses. For example, the mosquito-associated flavivirus may be selected from the group consisting of the Kamiti River virus, the Culex flavivirus, the Aedes flavivirus, the Nakiwogo virus, the Quang Binh virus, and the Cell Fusing Agent virus.

In some embodiments, the backbone may be selected from a flavivirus that has been adapted to a cell substrate, e.g., acceptable for use in preparing vaccines for human or veterinary use. For example, the backbone may be selected from a flavivirus that has been adapted to Vero, Madin Darby Canine kidney (MDCK), A549, fetal rhesus lung (FRhL), MRC5, human embryonic kidney (HEK293), primary dog kidney, primary rabbit kidney, xenopus oocytes, or chick embryo cells. In some embodiments, the backbone is selected from a flavivirus adapted to a cell substrate such that an increase in yield of the virus is at least about 2.5-fold as compared to the flavivirus before adaptation. In additional embodiments, the backbone may comprise at least one amino acid modification in a non-structural protein. In certain embodiments, the non-structural protein is NS1. For example, the NS1 protein may comprise a substitution at an amino acid position corresponding to the proline 315 residue of the Rio Bravo virus NS1 (SEQ ID NO: 1). In certain specific embodiments, the proline 315 residue of the NS1 protein is replaced with a serine residue. In certain embodiments, the non-structural protein is NS3. For example, the NS3 protein may comprise a substitution at an amino acid position corresponding to the isoleucine 555 residue of the Rio Bravo virus NS3 (SEQ ID NO: 2). In certain specific embodiments, the isoleucine 555 residue of the NS3 protein is replaced with a threonine residue.

In some embodiments, the chimeric flavivirus vector may comprise at least one nucleotide deletion in the 3′ non-coding region (NCR). For example, the 3′ non-coding region may comprise a nucleotide deletion at a nucleotide corresponding to the thymine at position 10692 of the Rio Bravo virus genome.

In one embodiment, the chimeric flavivirus vector may further comprise a signal sequence at the 3′ end of the capsid gene, e.g., a target for a host cell furin enzyme which cleaves the translated open reading frame at that site. In another embodiment, the chimeric flavivirus vector may be within a plasmid comprising at least one cytomegalovirus (CMV) promoter, e.g., operably linked to the chimeric flavivirus vector. In yet another embodiment, the chimeric flavivirus vector may be within a plasmid expressing at least one hepatitis δ virus ribozyme. In yet another embodiment, the chimeric flavivirus vector may be within a plasmid comprising at least one SV40 polyadenylation site. In yet another embodiment, the chimeric flavivirus vector may comprise one or more intron sequences. For example, in one embodiment, the chimeric flavivirus vector may comprise an intron at the junction of the envelope (E) and the non-structural gene 1 (NS1). In another embodiment, the chimeric flavivirus vector may comprise an intron immediately after a nucleotide corresponding to position 9742 of the Rio Bravo virus genome.

In some embodiments, the chimeric flavivirus vector encodes a chimeric flavivirus capable of replicating in mammalian cells to at least about 10⁸ PFU/mL. In a further embodiment, the chimeric flavivirus vector encodes a chimeric flavivirus capable of replicating in mammalian cells to at least about 10⁹ PFU/mL. In additional embodiments, the chimeric flavivirus vector encodes a chimeric flavivirus capable of replicating in insect cells, but not capable of replicating in human cells.

In yet another aspect, the invention provides a chimeric flavivirus vector encoding a structural protein from a first flavivirus with a low level of replication in a cell and a backbone from a second flavivirus with a high level of replication in the cell, wherein the structural protein is an envelope protein and the backbone comprises the capsid protein, the non-structural proteins and the 3′ and 5′ noncoding termini, and wherein the second flavivirus is a flavivirus that does not cause cytopathic effect (CPE) when growing in a cell culture. In some embodiments, the second flavivirus allows more than one harvesting of a cell culture fluid from a cell culture when growing in the cell culture. In one embodiment, the first flavivirus with a low level of replication in a cell is selected from the group consisting of dengue virus and yellow fever virus. In another embodiment, the first flavivirus is a dengue virus selected from the group consisting of dengue type 1 (DEN1) virus, dengue type 2 (DEN2) virus, dengue type 3 (DEN3) virus, and dengue type 4 (DEN4) virus.

In certain embodiments, the second flavivirus is a flavivirus selected from the Rio Bravo taxonomic group. For example, the flavivirus of the Rio Bravo taxonomic group may be selected from the group consisting of the Rio Bravo (sensu stricto) virus, the Montana Myotis Leukoencephalitis virus, the Dakar Bat virus, the Phnom Penh Bat virus, the Carey Island virus, and the Bukalasa Bat virus.

In still other embodiments, the second flavivirus with a high level of replication in the cell is a flavivirus selected from the mosquito-associated flaviviruses. For example, the mosquito-associated flavivirus may be selected from the group consisting of the Kamiti River virus, the Culex flavivirus, the Aedes flavivirus, the Nakiwogo virus, the Quang Binh virus, and the Cell Fusing Agent virus.

In some embodiments, the second flavivirus may be selected from a flavivirus that has been adapted to a cell substrate. For example, the second flavivirus may be selected from a flavivirus that has been adapted to Vero, Madin Darby Canine kidney (MDCK), A549, fetal rhesus lung (FRhL), MRC5, human embryonic kidney (HEK293), primary dog kidney, primary rabbit kidney, xenopus oocytes, or chick embryo cells. In some embodiments, the backbone is selected from a flavivirus adapted to a cell substrate such that an increase in yield of the virus is at least about 2.5-fold as compared to the flavivirus before adaptation. In additional embodiments, the second flavivirus may comprise at least one amino acid modification in a non-structural protein. In certain embodiments, the non-structural protein is NS1. For example, the NS1 protein may comprise a substitution at an amino acid position corresponding to the proline 315 residue of the Rio Bravo virus NS1 (SEQ ID NO: 1). In certain specific embodiments, the proline 315 residue of the NS1 protein is replaced with a serine residue. In certain embodiments, the non-structural protein is NS3. For example, the NS3 protein may comprise a substitution at an amino acid position corresponding to the isoleucine 555 residue of the Rio Bravo virus NS3 (SEQ ID NO: 2). In certain specific embodiments, the isoleucine 555 residue of the NS3 protein is replaced with a threonine residue.

In some embodiments, the chimeric flavivirus vector may comprise at least one nucleotide deletion in the 3′ non-coding region (NCR). For example, the 3′ non-coding region may comprise a nucleotide deletion at a nucleotide corresponding to the thymine at position 10692 of the Rio Bravo virus genome.

In one embodiment, the chimeric flavivirus vector may further comprise a signal sequence at the 3′ end of the capsid gene. In another embodiment, the chimeric flavivirus vector may be within a plasmid comprising at least one cytomegalovirus (CMV) promoter, e.g., operably linked to the chimeric flavivirus vector. In yet another embodiment, the chimeric flavivirus vector may be within a plasmid expressing at least one hepatitis δ virus ribozyme. In yet another embodiment, the chimeric flavivirus vector may be within a plasmid comprising at least one SV40 polyadenylation site. In yet another embodiment, the chimeric flavivirus vector may comprise one or more intron sequences. For example, in one embodiment, the chimeric flavivirus vector may comprise an intron at the junction of the envelope (E) and the non-structural gene 1 (NS1). In another embodiment, the chimeric flavivirus vector may comprise an intron immediately after a nucleotide corresponding to position 9742 of the Rio Bravo virus genome.

In some embodiments, the chimeric flavivirus vector encodes a chimeric flavivirus capable of replicating in mammalian cells to at least about 10⁸ PFU/mL. In a further embodiment, the chimeric flavivirus vector encodes a chimeric flavivirus capable of replicating in mammalian cells to at least about 10⁹ PFU/mL. In additional embodiments, the chimeric flavivirus vector encodes a chimeric flavivirus capable of replicating in insect cells, but not capable of replicating in human cells.

In yet another aspect, the invention provides a chimeric flavivirus vector encoding a structural protein from a first flavivirus with a low level of replication in a cell and a backbone from a second flavivirus with a high level of replication in the cell, wherein the structural protein is an envelope protein and the backbone comprises the capsid protein, the non-structural proteins and the 3′ and 5′ noncoding termini, and wherein the second flavivirus is a flavivirus that does not cause an elevation in extracellular DNA release as compared to a mock infection when growing in a cell culture, and as measured at day 3 post-infection. In one embodiment, the first flavivirus with a low level of replication in a cell is selected from the group consisting of dengue virus and yellow fever virus. In another embodiment, the first flavivirus is a dengue virus selected from the group consisting of dengue type 1 (DEN1) virus, dengue type 2 (DEN2) virus, dengue type 3 (DEN3) virus, and dengue type 4 (DEN4) virus.

In certain embodiments, the second flavivirus with a high level of replication in the cell is a flavivirus selected from the Rio Bravo taxonomic group. For example, the flavivirus of the Rio Bravo taxonomic group may be selected from the group consisting of the Rio Bravo (sensu stricto) virus, the Montana Myotis Leukoencephalitis virus, the Dakar Bat virus, the Phnom Penh Bat virus, the Carey Island virus, and the Bukalasa Bat virus.

In still other embodiments, the second flavivirus with a high level of replication in the cell is a flavivirus selected from the mosquito-associated flaviviruses. For example, the mosquito-associated flavivirus may be selected from the group consisting of the Kamiti River virus, the Culex flavivirus, the Aedes flavivirus, the Nakiwogo virus, the Quang Binh virus, and the Cell Fusing Agent virus.

In some embodiments, the second flavivirus may be selected from a flavivirus that has been adapted to a cell substrate. For example, the second flavivirus may be selected from a flavivirus that has been adapted to Vero, Madin Darby Canine kidney (MDCK), A549, fetal rhesus lung (FRhL), MRC5, human embryonic kidney (HEK293), primary dog kidney, primary rabbit kidney, xenopus oocytes, or chick embryo cells. In some embodiments, the backbone is selected from a flavivirus adapted to a cell substrate such that an increase in yield of the virus is at least about 2.5-fold as compared to the flavivirus before adaptation. In additional embodiments, the second flavivirus may comprise at least one amino acid modification in a non-structural protein. In certain embodiments, the non-structural protein is NS1. For example, the NS1 protein may comprise a substitution at an amino acid position corresponding to the proline 315 residue of the Rio Bravo virus NS1 (SEQ ID NO: 1). In certain specific embodiments, the proline 315 residue of the NS1 protein is replaced with a serine residue. In certain embodiments, the non-structural protein is NS3. For example, the NS3 protein may comprise a substitution at an amino acid position corresponding to the isoleucine 555 residue of the Rio Bravo virus NS3 (SEQ ID NO: 2). In certain specific embodiments, the isoleucine 555 residue of the NS3 protein is replaced with a threonine residue.

In some embodiments, the chimeric flavivirus vector may comprise at least one nucleotide deletion in the 3′ non-coding region (NCR). For example, the 3′ non-coding region may comprise a nucleotide deletion at a nucleotide corresponding to the thymine at position 10692 of the Rio Bravo virus genome.

In one embodiment, the chimeric flavivirus vector may further comprise a signal sequence at the 3′ end of the capsid gene. In another embodiment, the chimeric flavivirus vector may be within a plasmid comprising at least one cytomegalovirus (CMV) promoter, e.g., operably linked to the chimeric flavivirus vector. In yet another embodiment, the chimeric flavivirus vector may be within a plasmid expressing at least one hepatitis δ virus ribozyme. In yet another embodiment, the chimeric flavivirus vector may be within a plasmid comprising at least one SV40 polyadenylation site. In yet another embodiment, the chimeric flavivirus vector may comprise one or more intron sequences. For example, in one embodiment, the chimeric flavivirus vector may comprise an intron at the junction of the envelope (E) and the non-structural gene 1 (NS1). In another embodiment, the chimeric flavivirus vector may comprise an intron immediately after a nucleotide corresponding to position 9742 of the Rio Bravo virus genome.

In some embodiments, the chimeric flavivirus vector encodes a chimeric flavivirus capable of replicating in mammalian cells to at least about 10⁸ PFU/mL. In a further embodiment, the chimeric flavivirus vector encodes a chimeric flavivirus capable of replicating in mammalian cells to at least about 10⁹ PFU/mL. In additional embodiments, the chimeric flavivirus vector encodes a chimeric flavivirus capable of replicating in insect cells, but not capable of replicating in human cells.

In yet another aspect, the invention provides a chimeric flavivirus encoded by a chimeric flavivirus vector. In various embodiments described herein, the chimeric flavivirus is encoded by a chimeric flavivirus vector, wherein the chimeric flavivirus vector encodes a structural protein from a first flavivirus with a low level of replication in a cell and a backbone from a second flavivirus with a high level of replication in the cell. Alternatively the chimeric flavivirus is encoded by a chimeric flavivirus vector, wherein the chimeric flavivirus vector encodes an envelope protein from a first flavivirus with a low level of replication in a cell and one or more non-structural proteins from a second flavivirus with a high level of replication in the cell.

In one embodiment, the structural protein is an envelope protein. In another embodiment, the structural protein comprises an envelope protein and a pre-membrane protein. In some embodiments, the backbone comprises a capsid protein, the non-structural proteins, and the 3′ and 5′ non-coding termini. In one embodiment, the first flavivirus with a low level of replication in a cell is selected from the group consisting of dengue virus and yellow fever virus. In another embodiment, the first flavivirus is a dengue virus selected from the group consisting of dengue type 1 (DEN1) virus, dengue type 2 (DEN2) virus, dengue type 3 (DEN3) virus, and dengue type 4 (DEN4) virus.

In certain embodiments, the second flavivirus with a high level of replication in the cell is a flavivirus selected from the Rio Bravo taxonomic group. For example, the flavivirus of the Rio Bravo taxonomic group may be selected from the group consisting of the Rio Bravo (sensu stricto) virus, the Montana Myotis Leukoencephalitis virus, the Dakar Bat virus, the Phnom Penh Bat virus, the Carey Island virus, and the Bukalasa Bat virus.

In certain other embodiments, the second flavivirus with a high level of replication in the cell is a flavivirus selected from the Uganda S taxonomic group. For example, the flavivirus of the Uganda S taxonomic group may be selected from the group consisting of the Uganda S virus, the Banzi virus, and the Jugra virus.

In still other embodiments, the second flavivirus with a high level of replication in the cell is a flavivirus selected from the mosquito-associated flaviviruses. For example, the mosquito-associated flavivirus may be selected from the group consisting of the Kamiti River virus, the Culex flavivirus, the Aedes flavivirus, the Nakiwogo virus, the Quang Binh virus, and the Cell Fusing Agent virus.

In some embodiments, the backbone may be selected from a flavivirus that has been adapted to a cell substrate. For example, the backbone may be selected from a flavivirus that has been adapted to Vero, Madin Darby Canine kidney (MDCK), A549, fetal rhesus lung (FRhL), MRC5, human embryonic kidney (HEK293), primary dog kidney, primary rabbit kidney, xenopus oocytes, or chick embryo cells. In some embodiments, the backbone is selected from a flavivirus adapted to a cell substrate such that an increase in yield of the virus is at least about 2.5-fold as compared to the flavivirus before adaptation. In additional embodiments, the backbone may comprise at least one amino acid modification in a non-structural protein. In certain embodiments, the non-structural protein is NS1. For example, the NS1 protein may comprise a substitution at an amino acid position corresponding to the proline 315 residue of the Rio Bravo virus NS1 (SEQ ID NO: 1). In certain specific embodiments, the proline 315 residue of the NS1 protein is replaced with a serine residue. In certain embodiments, the non-structural protein is NS3. For example, the NS3 protein may comprise a substitution at an amino acid position corresponding to the isoleucine 555 residue of the Rio Bravo virus NS3 (SEQ ID NO: 2). In certain specific embodiments, the isoleucine 555 residue of the NS3 protein is replaced with a threonine residue.

In some embodiments, the chimeric flavivirus vector may comprise at least one nucleotide deletion in the 3′ non-coding region (NCR). For example, the 3′ non-coding region may comprise a nucleotide deletion at a nucleotide corresponding to the thymine at position 10692 of the Rio Bravo virus genome.

In one embodiment, the chimeric flavivirus vector may further comprise a signal sequence at the 3′ end of the capsid gene. In another embodiment, the chimeric flavivirus vector may be within a plasmid comprising at least one cytomegalovirus (CMV) promoter, e.g., operably linked to the chimeric flavivirus vector. In yet another embodiment, the chimeric flavivirus vector may be within a plasmid expressing at least one hepatitis δ virus ribozyme. In yet another embodiment, the chimeric flavivirus vector may be within a plasmid comprising at least one SV40 polyadenylation site. In yet another embodiment, the chimeric flavivirus vector may comprise one or more intron sequences. For example, in one embodiment, the chimeric flavivirus vector may comprise an intron at the junction of the envelope (E) and the non-structural gene 1 (NS1). In another embodiment, the chimeric flavivirus vector may comprise an intron immediately after a nucleotide corresponding to position 9742 of the Rio Bravo virus genome.

In some embodiments, the chimeric flavivirus vector encodes a chimeric flavivirus capable of replicating in mammalian cells to at least about 10⁸ PFU/mL. In a further embodiment, the chimeric flavivirus vector encodes a chimeric flavivirus capable of replicating in mammalian cells to at least about 10⁹ PFU/mL. In additional embodiments, the chimeric flavivirus vector encodes a chimeric flavivirus capable of replicating in insect cells, but not capable of replicating in human cells.

In yet another aspect, the invention provides an inactivated chimeric flavivirus encoded by a chimeric flavivirus vector described herein or alternatively derived from a chimeric flavivirus comprising a structural protein, e.g., envelope protein and optionally a membrane protein from a first flavivirus with a low level of replication in a cell and 1) a capsid protein from a second flavivirus with a high level of replication in the cell, 2) an RNA genome comprising a sequence encoding one or more or all of the non-structural proteins, e.g., NS1, NS2A, NS2B, NS3, NS4A, NS4B, and NS5 from a second flavivirus with a high level of replication in the cell, or 3) an RNA genome with both 3′ and 5′ termini of the genome from a second flavivirus with a high level of replication in the cell, or a combination thereof. In certain embodiments, the chimeric flavivirus, e.g., infectious viral particles may be inactivated with a method selected from the group consisting of chemical inactivation, high pressure inactivation, ultraviolet radiation, and gamma radiation. In an exemplary embodiment, the chimeric flavivirus is inactivated using chemical inactivation. For example, the method of chemical inactivation may comprise exposure of the flavivirus to one or more agents selected from the group consisting of β-propiolactone, formalin, aziridines, hydrogen peroxide, organic solvents, and ascorbic acid.

In yet another aspect, the invention provides a vaccine comprising an inactivated chimeric flavivirus described herein. In various embodiments described herein, the vaccine comprises an inactivated chimeric flavivirus, wherein the inactivated chimeric flavivirus is derived from a chimeric flavivirus vector that encodes a structural protein from a first flavivirus with a low level of replication in a cell and a backbone from a second flavivirus with a high level of replication in the cell. In one embodiment, the structural protein is an envelope protein. In another embodiment, the structural protein comprises an envelope protein and a pre-membrane protein. In some embodiments, the backbone comprises a capsid protein, the non-structural proteins, and the 3′ and 5′ non-coding termini. In one embodiment, the first flavivirus with a low level of replication in a cell is selected from the group consisting of dengue virus and yellow fever virus. In another embodiment, the first flavivirus is a dengue virus selected from the group consisting of dengue type 1 (DEN1) virus, dengue type 2 (DEN2) virus, dengue type 3 (DEN3) virus, and dengue type 4 (DEN4) virus.

In certain embodiments, the second flavivirus with a high level of replication in the cell is a flavivirus selected from the Rio Bravo taxonomic group. For example, the flavivirus of the Rio Bravo taxonomic group may be selected from the group consisting of the Rio Bravo (sensu stricto) virus, the Montana Myotis Leukoencephalitis virus, the Dakar Bat virus, the Phnom Penh Bat virus, the Carey Island virus, and the Bukalasa Bat virus.

In certain other embodiments, the second flavivirus with a high level of replication in the cell is a flavivirus selected from the Uganda S taxonomic group. For example, the flavivirus of the Uganda S taxonomic group may be selected from the group consisting of the Uganda S virus, the Banzi virus, and the Jugra virus.

In still other embodiments, the second flavivirus with a high level of replication in the cell is a flavivirus selected from the mosquito-associated flaviviruses. For example, the mosquito-associated flavivirus may be selected from the group consisting of the Kamiti River virus, the Culex flavivirus, the Aedes flavivirus, the Nakiwogo virus, the Quang Binh virus, and the Cell Fusing Agent virus.

In some embodiments, the backbone may be selected from a flavivirus that has been adapted to a cell substrate. For example, the backbone may be selected from a flavivirus that has been adapted to Vero, Madin Darby Canine kidney (MDCK), A549, fetal rhesus lung (FRhL), MRC5, human embryonic kidney (HEK293), primary dog kidney, primary rabbit kidney, xenopus oocytes, or chick embryo cells. In some embodiments, the backbone is selected from a flavivirus adapted to a cell substrate such that an increase in yield of the virus is at least about 2.5-fold as compared to the flavivirus before adaptation. In additional embodiments, the backbone may comprise at least one amino acid modification in a non-structural protein. In certain embodiments, the non-structural protein is NS1. For example, the NS1 protein may comprise a substitution at an amino acid position corresponding to the proline 315 residue of the Rio Bravo virus NS1 (SEQ ID NO: 1). In certain specific embodiments, the proline 315 residue of the NS1 protein is replaced with a serine residue. In certain embodiments, the non-structural protein is NS3. For example, the NS3 protein may comprise a substitution at an amino acid position corresponding to the isoleucine 555 residue of the Rio Bravo virus NS3 (SEQ ID NO: 2). In certain specific embodiments, the isoleucine 555 residue of the NS3 protein is replaced with a threonine residue.

In some embodiments, the chimeric flavivirus vector may comprise at least one nucleotide deletion in the 3′ non-coding region (NCR). For example, the 3′ non-coding region may comprise a nucleotide deletion at a nucleotide corresponding to the thymine at position 10692 of the Rio Bravo virus genome.

In one embodiment, the chimeric flavivirus vector may further comprise a signal sequence at the 3′ end of the capsid gene. In another embodiment, the chimeric flavivirus vector may be within a plasmid comprising at least one cytomegalovirus (CMV) promoter, e.g., operably linked to the chimeric flavivirus vector. In yet another embodiment, the chimeric flavivirus vector may be within a plasmid expressing at least one hepatitis δ virus ribozyme. In yet another embodiment, the chimeric flavivirus vector may be within a plasmid comprising at least one SV40 polyadenylation site. In yet another embodiment, the chimeric flavivirus vector may comprise one or more intron sequences. For example, in one embodiment, the chimeric flavivirus vector may comprise an intron at the junction of the envelope (E) and the non-structural gene 1 (NS1). In another embodiment, the chimeric flavivirus vector may comprise an intron immediately after a nucleotide corresponding to position 9742 of the Rio Bravo virus genome.

In some embodiments, the chimeric flavivirus vector encodes a chimeric flavivirus capable of replicating in mammalian cells to at least about 10⁸ PFU/mL. In a further embodiment, the chimeric flavivirus vector encodes a chimeric flavivirus capable of replicating in mammalian cells to at least about 10⁹ PFU/mL.

In yet another aspect, the invention provides a vaccine comprising an inactivated chimeric flavivirus. In one embodiment, the inactivated chimeric flavivirus is derived from a chimeric flavivirus comprising a structural protein, e.g., envelope protein or membrane protein or both from a first flavivirus with a low level of replication in a cell and 1) a capsid protein from a second flavivirus with a high level of replication in the cell, 2) an RNA genome comprising a sequence encoding one or more or all of the non-structural proteins, e.g., NS1, NS2A, NS2B, NS3, NS4A, NS4B, and NS5 from a second flavivirus with a high level of replication in the cell, or 3) an RNA genome with both 3′ and 5′ termini of the genome from a second flavivirus with a high level of replication in the cell, or a combination thereof. In one embodiment, the first flavivirus with a low level of replication in a cell is selected from the group consisting of dengue virus and yellow fever virus. In another embodiment, the first flavivirus is a dengue virus selected from the group consisting of dengue type 1 (DEN1) virus, dengue type 2 (DEN2) virus, dengue type 3 (DEN3) virus, and dengue type 4 (DEN4) virus.

In certain embodiments, the second flavivirus with a high level of replication in the cell is a flavivirus selected from the Rio Bravo taxonomic group. For example, the flavivirus of the Rio Bravo taxonomic group may be selected from the group consisting of the Rio Bravo (sensu stricto) virus, the Montana Myotis Leukoencephalitis virus, the Dakar Bat virus, the Phnom Penh Bat virus, the Carey Island virus, and the Bukalasa Bat virus.

In certain other embodiments, the second flavivirus with a high level of replication in the cell is a flavivirus selected from the Uganda S taxonomic group. For example, the flavivirus of the Uganda S taxonomic group may be selected from the group consisting of the Uganda S virus, the Banzi virus, and the Jugra virus.

In still other embodiments, the second flavivirus with a high level of replication in the cell is a flavivirus selected from the mosquito-associated flaviviruses. For example, the mosquito-associated flavivirus may be selected from the group consisting of the Kamiti River virus, the Culex flavivirus, the Aedes flavivirus, the Nakiwogo virus, the Quang Binh virus, and the Cell Fusing Agent virus.

In still yet other embodiments, the second flavivirus with a high level of replication in the cell is a West Nile virus.

In some embodiments, the second flavivirus may be selected from a flavivirus that has been adapted to a cell substrate. For example, the second flavivirus may be selected from a flavivirus that has been adapted to Vero, Madin Darby Canine kidney (MDCK), A549, fetal rhesus lung (FRhL), MRC5, human embryonic kidney (HEK293), primary dog kidney, primary rabbit kidney, xenopus oocytes, or chick embryo cells. In some embodiments, the backbone is selected from a flavivirus adapted to a cell substrate such that an increase in yield of the virus is at least about 2.5-fold as compared to the flavivirus before adaptation. In additional embodiments, the second flavivirus may comprise at least one amino acid modification in a non-structural protein. In certain embodiments, the non-structural protein is NS1. For example, the NS1 protein may comprise a substitution at an amino acid position corresponding to the proline 315 residue of the Rio Bravo virus NS1 (SEQ ID NO: 1). In certain specific embodiments, the proline 315 residue of the NS1 protein is replaced with a serine residue. In certain embodiments, the non-structural protein is NS3. For example, the NS3 protein may comprise a substitution at an amino acid position corresponding to the isoleucine 555 residue of the Rio Bravo virus NS3 (SEQ ID NO: 2). In certain specific embodiments, the isoleucine 555 residue of the NS3 protein is replaced with a threonine residue.

In some embodiments, the chimeric flavivirus is capable of replicating in mammalian cells to at least about 10⁸ PFU/mL. In a further embodiment, the chimeric flavivirus is capable of replicating in mammalian cells to at least about 10⁹ PFU/mL. In additional embodiments, the chimeric flavivirus is capable of replicating in insect cells, but not capable of replicating in human cells.

In various embodiments described herein, vaccines of the invention may additionally comprise an adjuvant. For example, a vaccine described herein may comprise an adjuvant selected from the group consisting of aluminum hydroxide, MF59, saponin, lipid A, iscomatrix, and immunostimulatory oligonucleotides.

In yet another aspect, the invention provides a vaccine comprising a live, chimeric flavivirus described herein. In various embodiments described herein, the vaccine comprises a live, chimeric flavivirus encoded by the chimeric flavivirus described herein and/or comprising a structural protein from a first flavivirus with a low level of replication in a cell and 1) a capsid protein from a second flavivirus with a high level of replication in the cell, 2) an RNA genome comprising a sequence encoding one or more or all of the non-structural proteins, e.g., NS1, NS2A, NS2B, NS3, NS4A, NS4B, and NS5 from a second flavivirus with a high level of replication in the cell, or 3) an RNA genome with both 3′ and 5′ termini of the genome from a second flavivirus with a high level of replication in the cell, or a combination thereof. In one embodiment, the structural protein is an envelope protein. In another embodiment, the structural protein comprises an envelope protein and a pre-membrane protein. In one embodiment, the first flavivirus with a low level of replication in a cell is selected from the group consisting of dengue virus and yellow fever virus. In another embodiment, the first flavivirus is a dengue virus selected from the group consisting of dengue type 1 (DEN1) virus, dengue type 2 (DEN2) virus, dengue type 3 (DEN3) virus, and dengue type 4 (DEN4) virus.

In certain embodiments, the second flavivirus with a high level of replication in the cell is a flavivirus selected from the mosquito-associated flaviviruses. For example, the mosquito-associated flavivirus may be selected from the group consisting of the Kamiti River virus, the Culex flavivirus, the Aedes flavivirus, the Nakiwogo virus, the Quang Binh virus, and the Cell Fusing Agent virus.

In some embodiments, the backbone may be selected from a flavivirus that has been adapted to a cell substrate. For example, the backbone may be selected from a flavivirus that has been adapted to C6/36 Aedes albopictus mosquito cells, u4.4 cells, High Five™ cells, Schneider's Drosophila cell line 2, Spodoptera frugiperda SF9 cells, Anopheles albimanus cells, Anopheles gambiae cells, Culex tarsalis cells, Phlebotomus papatasi cells, and C7-10 cells. In some embodiments, the backbone is selected from a flavivirus adapted to a cell substrate such that an increase in yield of the virus is at least about 2.5-fold as compared to the flavivirus before adaptation.

In some embodiments, the chimeric flavivirus is capable of replicating in mammalian cells to at least about 10⁸ PFU/mL. In a further embodiment, the chimeric flavivirus is capable of replicating in mammalian cells to at least about 10⁹ PFU/mL. In additional embodiments, the chimeric flavivirus vector encodes a chimeric flavivirus capable of replicating in insect cells, but not capable of replicating in human cells.

In additional aspects, the invention provides vaccines comprising one or more chimeric flavivirus vectors described herein. In one embodiment, the chimeric flavivirus vector encodes a structural protein from a first flavivirus with a low level of replication in a cell and a backbone from a second flavivirus with a high level of replication in the cell. In another embodiment, the chimeric flavivirus vector encodes an envelope protein from a first flavivirus with a low level of replication in a cell and one or more non-structural proteins from a second flavivirus with a high level of replication in the cell. In yet another embodiment, the chimeric flavivirus vector encodes a structural protein from a first flavivirus with a low level of replication in a cell and a backbone from a second flavivirus with a high level of replication in the cell, wherein the structural protein is an envelope protein and the backbone comprises the capsid protein, the non-structural proteins and the 3′ and 5′ noncoding termini, and wherein the second flavivirus is a flavivirus does not cause cytopathic effect (CPE) when growing in a cell culture. In still yet another embodiment, the chimeric flavivirus vector encodes a structural protein from a first flavivirus with a low level of replication in a cell and a backbone from a second flavivirus with a high level of replication in the cell, wherein the structural protein is an envelope protein and the backbone comprises the capsid protein, the non-structural proteins and the 3′ and 5′ noncoding termini, and wherein the second flavivirus is a flavivirus that does not cause an elevation in extracellular DNA release as compared to a mock infection when growing in a cell culture and as measured at day 3 post-infection.

In various embodiments described herein, the vaccine may be formulated for parenteral or mucosal, e.g., oral administration.

In yet another aspect, the invention provides a method for inducing an immune response in a subject. In one embodiment, the method comprises administration of a chimeric flavivirus encoded by a chimeric flavivirus vector described herein. In some embodiments, the chimeric flavivirus is an inactivated chimeric flavivirus. In another embodiment, the chimeric flavivirus is a live chimeric flavivirus. In a further embodiment, the inactivated chimeric flavivirus is administered in conjunction with an adjuvant. In other embodiments, the method comprises administration of a chimeric flavivirus vector described herein. In a further embodiment, the chimeric flavivirus vector is administered in conjunction with an adjuvant. In some embodiments, the invention provides a method for inducing an immune response to a dengue virus in a subject. In alternative embodiments, the invention provides a method for inducing an immune response to a yellow fever virus in a subject.

In yet another aspect, the invention provides a method for vaccination of a subject. In one embodiment, the method comprises administration of a chimeric flavivirus encoded by a chimeric flavivirus vector described herein. In one embodiment, the chimeric flavivirus is an inactivated chimeric flavivirus. In another embodiment, the chimeric flavivirus is a live chimeric flavivirus. In a further embodiment, the inactivated flavivirus is administered in conjunction with an adjuvant. In other embodiments, the method comprises administration of a chimeric flavivirus vector described herein. In a further embodiment, the chimeric flavivirus vector is administered in conjunction with an adjuvant. In some embodiments, the invention provides a method for vaccination of a subject to prevent infection with the dengue virus. In alternative embodiments, the invention provides a method for vaccination of a subject to prevent infection with the yellow fever virus.

Additional aspects and embodiments of the invention will be apparent from the detailed description that follows.

### BRIEF DESCRIPTION OF THE DRAWINGS

FIG. 1 is a diagram which illustrates the phylogenetic relationships of members of the Flavivirus genus. Members of the Rio Bravo taxonomic group, the Uganda S taxonomic group, and the West Nile virus are circled.

FIG. 2A illustrates the growth of selected flaviviruses in Vero cells (P2, no adaptation).

FIG. 2B illustrates the (Top) Banzi virus on day 3, exhibiting 4+ cytopathic effects (CPE) and the (Bottom) Rio Bravo virus on day 5, exhibiting minimal cytopathic effects (CPE).

FIG. 3 illustrates the growth kinetics of the Rio Bravo virus before passage 1 (P1) and after adaptation by serial passage (P10).

FIG. 4 illustrates the growth kinetics (Vero WHO 10-87 cells) of small plaque versus large plaque population of Rio Bravo virus. The plaque populations differed in genomic sequence as described in the text.

FIG. 5 illustrates a comparison of growth kinetics of dengue-2 strains belonging to Lineage I and II isolated from humans in Peru.

FIG. 6 illustrates the growth kinetics of individual dengue-2 strains in Vero cells. Some strains (in Lineage II) grew to relatively high titer (>10⁶ PFU/mL). Strain FPI00174 was selected as a donor strain for construction of chimeric virus.

FIGS. 7A through 7E illustrate a cloning strategy for the construction of a chimeric flavivirus vector comprising a Rio Bravo virus backbone and a structural protein from a dengue virus.

### DETAILED DESCRIPTION

The present invention is based, in part, on the discovery that one or more structural proteins from a first flavivirus with low level of replication in cells can be integrated into a second flavivirus with a high level of replication in cells, e.g., one or more structural proteins of a first flavivirus can be used to replace one or more structural proteins of a second flavivirus. Accordingly, the present invention provides chimeric flavivirus vectors, chimeric flaviviruses encoded thereof, and vaccines containing chimeric flavivirus vectors or chimeric flavivirus(es). In addition, the present invention also provides methods of vaccination or inducing an immune response using the vaccines of the present invention.

### Chimeric Flavivirus Vectors

In one aspect, the invention provides a chimeric flavivirus vector encoding a structural protein from a first flavivirus with a low level of replication in a cell and a backbone from a second flavivirus with a high level of replication in the cell.

As used herein, “chimeric flavivirus vector” refers to any polynucleotide containing or comprising the desired nucleotide sequence, e.g., the nucleotide sequence encoding the chimeric flavivirus of the present invention. The polynucleotide can be DNA, RNA, cDNA and can include naturally existing nucleotides or any modified nucleotides. The polynucleotide can be within any suitable construct, e.g., for cloning, infection, replication, expression, etc. In some embodiments, the polynucleotide is within a plasmid, e.g., for cloning and/or infection. In some other embodiments, the polynucleotide is within a viral genome. In yet some other embodiments, the polypeptide is independent of any construct.

As used herein, “a low level of replication in a cell” as it refers to a flavivirus' ability to replicate in a cell, relates to a wild-type flavivirus which replicates to maximum levels of less than about 10⁶, 10⁷ or 10⁸ plaque-forming units (PFUs)/mL in cell culture. In certain embodiments, flaviviruses with a low level of replication in a cell include dengue viruses and yellow fever viruses.

As used herein, “a high level of replication in a cell” as it refers to a flavivirus' ability to replicate in a cell, relates to a wild-type flavivirus which replicates to maximum levels of at least about 10⁸, 10⁹, or 10¹⁰ plaque-forming units (PFUs)/mL in cell culture. In certain embodiments, flaviviruses with a high level of replication in a cell include West Nile viruses, Japanese encephalitis viruses, tick-borne encephalitis viruses, flaviviruses of the Rio Bravo taxonomic group, flaviviruses of the Uganda S taxonomic group, and mosquito-associated flaviviruses.

As used herein, “a backbone” refers to a structural component of a virus genome. In some embodiments, it includes sequences encoding one or more or all non-structural components of a virus genome, e.g., NS1, NS2A, NS2B, NS3, NS4, NS4B, and NS5. In some other embodiments, it includes sequences encoding all non-structural components as well as a capsid protein. In some other embodiments, it includes sequences encoding all non-structural components, capsid protein as well as 3′ and 5′ non-coding termini of a virus. In some other embodiments, it includes sequences encoding all components of a virus other than its envelope protein and optionally the membrane protein. In yet some other embodiments, it includes naturally existing sequences, modified sequences with natural or non-natural nucleotides. In still some other embodiments, it includes sequences derived from the naturally existing sequences, e.g., it includes sequences found in naturally existing sequences with mutations such as deletions, additions, substitutions so long as products encoded thereof function equivalently or better than the products encoded by naturally existing sequences.

The invention is directed, in part, to methods of growing high levels of a flavivirus having the envelope protein (E) (containing protective, neutralizing epitopes), fragments thereof, variants thereof, or derivatives thereof and optionally membrane protein, fragments thereof, variants thereof, or derivatives thereof from a first flavivirus with a low level of replication in a cell in cell culture which can either be rendered safe (non-replicating) by chemical inactivation, or by use of a virus incapable of replicating in humans. The methods of the invention employ the construction of a chimeric flavivirus vector that comprises a sequence encoding the envelope and optionally pre-membrane protein of a first flavivirus with a low level of replication in a cell. In some embodiments, the envelope and optionally pre-membrane protein of a first flavivirus when become part of the chimeric flavivirus contains epitopes in their natural conformation and the chimeric flavivirus is capable of replicating to a titer in acceptable cell cultures sufficient for manufacture of an inactivated vaccine.

In certain embodiments, the chimeric flavivirus vector encodes a structural protein from a first flavivirus with a low level of replication in a cell. In one embodiment, the structural protein is an envelope (E) protein, fragment thereof, variant thereof, or derivative thereof. In yet another embodiment, the chimeric flavivirus vector encodes an envelope protein, fragment thereof, variant thereof, or derivative thereof, and a pre-membrane protein, fragment thereof, variant thereof, or derivative thereof from a first flavivirus with a low level of replication in a cell. In an exemplary embodiment, the envelope protein, fragment thereof, variant thereof, or derivative thereof and the pre-membrane protein, fragment thereof, variant thereof, or derivative thereof are from the same flavivirus with a low level of replication in a cell. In an alternative embodiment, the envelope protein, fragment thereof, variant thereof, or derivative thereof, and the pre-membrane protein, fragment thereof, variant thereof, or derivative thereof are from a different flavivirus with a low level of replication in a cell, e.g., the pre-membrane protein, fragment thereof, variant thereof, or derivative thereof is from a second flavivirus with a high level of replication in a cell.

In some embodiments, the disclosure identifies specific genes (e.g., envelope, pre-membrane, non-structural, and/or capsid genes) useful in the compositions and methods of the disclosure. However, it will be recognized that absolute identity to such genes is not necessary. For example, changes in a particular gene or polynucleotide comprising a sequence encoding a polypeptide (e.g., an envelope and optionally a pre-membrane polypeptide) can be made and screened for the desired immunogenicity. Typically such changes comprise conservative mutations and silent mutations. Such modified or mutated polynucleotides and polypeptides can be screened for expression of a functional immunogenic polypeptide using methods known in the art.

Due to the inherent degeneracy of the genetic code, other polynucleotides which encode substantially the same or functionally equivalent polypeptides can also be used to clone and express the polynucleotides encoding such polypeptides.

As will be understood by those of skill in the art, it can be advantageous to modify a coding sequence to enhance its expression in a particular host. The genetic code is redundant with 64 possible codons, but most organisms typically use a subset of these codons. The codons that are utilized most often in a species are called optimal codons, and those not utilized very often are classified as rare or low-usage codons. Codons can be substituted to reflect the preferred codon usage of the host cell, in a process sometimes called “codon optimization” or “controlling for species codon bias.”

The coding regions encoding flavivirus polypeptides or fragments, variants, or derivatives thereof may be codon optimized. In some embodiments, the coding regions encoding a flavivirus envelope and/or pre-membrane polypeptide or fragment, variant, or derivative thereof are codon optimized Codon optimization is carried out by the methods well known in the art, for example, in certain embodiments codon-optimized coding regions encoding polypeptides of flavivirus, or nucleic acid fragments of such coding regions encoding fragments, variants, or derivatives thereof are optimized according to the codon usage of the particular host cell. In certain embodiments, the host cell is selected from the group consisting of Vero, Madin Darby Canine kidney (MDCK), A549, fetal rhesus lung (FRhL), MRC5, human embryonic kidney (HEK293), primary dog kidney, primary rabbit kidney, xenopus oocytes, or chick embryo cells.

Furthermore, those skilled in the art will recognize that, due to the degenerate nature of the genetic code, a variety of DNA compounds differing in their nucleotide sequences can be used to encode a given polypeptide of the disclosure (e.g., an envelope and/or pre-membrane polypeptide). The native DNA sequence encoding the polypeptides described herein are referenced merely to illustrate an embodiment of the disclosure, and the disclosure includes DNA compounds of any sequence that encode the amino acid sequences of the polypeptides utilized in the methods of the disclosure. In a similar fashion, a polypeptide can typically tolerate one or more amino acid substitutions, deletions, and insertions in its amino acid sequence without loss or significant loss of a desired immunogenicity. The disclosure includes such polypeptides with different amino acid sequences than the specific proteins described herein so long as the modified or variant polypeptides have the desired immunogenicity of the reference polypeptide. In certain embodiments, the polypeptides described herein (e.g., envelope and/or pre-membrane polypeptides) may be modified using known techniques to exhibit increased immunogenicity.

As described above, in some embodiments, the chimeric flavivirus vectors of the invention encode a structural protein from a first flavivirus with a low level of replication in a cell. In certain embodiments, the first flavivirus with a low level of replication in a cell is a dengue virus. In a further embodiment, the dengue virus may be selected from the group consisting of dengue type 1 (DEN1) virus, dengue type 2 (DEN2) virus, dengue type 3 (DEN3) virus, and dengue type 4 (DEN4) virus. As described herein, the present invention provides, in part, inactivated chimeric flaviviruses derived from chimeric flavivirus vectors comprising a sequence encoding an envelope protein and optionally a pre-membrane protein from a dengue virus for use in vaccines.

Not wishing to be limited to any technical details, in some embodiments the use of an inactivated, whole virion dengue vaccine provides a number of advantages over previous approaches, e.g., (a) the requirement of no more than two doses to achieve complete immunization (whereas three doses are required for the leading live vaccine); (b) no interference between non-replicating antigens in a tetravalent mixture; (c) 90-100% of subjects develop antibody to all four serotypes; and (d) since interference does not occur, the two doses of vaccine can be given at short intervals, e.g., 14, 21, or 28 days (whereas the leading live vaccine requires dosing spaced apart at approximately 3-6 months).

In certain other embodiments, the first flavivirus with a low level of replication in a cell is a yellow fever virus. As described herein, the present invention provides, in part, inactivated chimeric flaviviruses derived from chimeric flavivirus vectors comprising an envelope protein and/or pre-membrane protein from a yellow fever virus for use in vaccines.

Not wishing to be limited to any technical details, in some embodiments the use of an inactivated, whole virion yellow fever vaccine provides a key advantage over previous approaches which utilize a live, attenuated vaccine. In contrast to live, attenuated yellow fever vaccines which can be associated with adverse events including potentially serious and fatal adverse neurotropic and viscerotropic events, the use of an inactivated, whole virion yellow fever vaccine mitigates the potential for serious adverse events associated with the currently available live, attenuated vaccines.

In certain embodiments, the chimeric flavivirus vectors of the invention comprise a backbone from a second flavivirus with a high level of replication in the cell. In one embodiment, the backbone from a second flavivirus with a high level of replication in the cell comprises a capsid protein. In another embodiment, the backbone from a second flavivirus with a high level of replication in the cell comprises one or more non-structural proteins. In yet another embodiment, the backbone from a second flavivirus with a high level of replication in the cell comprises all non-structural proteins. In yet another embodiment, the backbone from a second flavivirus with a high level of replication in the cell comprises the 3′ and 5′ noncoding termini. In an exemplary embodiment, the backbone from a second flavivirus with a high level of replication in the cell comprises a capsid protein, one or more non-structural proteins, and the 3′ and 5′ noncoding termini. In another exemplary embodiment, the backbone from a second flavivirus with a high level of replication in the cell comprises a capsid protein, all non-structural proteins, and the 3′ and 5′ noncoding termini.

In certain embodiments, the second flavivirus is a flavivirus capable of high level replication in a cell and replicates without causing extensive cytopathic effects (CPE) in the cell, e.g., without causing substantial degenerative changes in the cell. For example, the second flavivirus with a high level of replication in a cell can be a flavivirus capable of high level replication in a cell as well as allowing more than one harvest of cell culture fluid, e.g., multiple harvest of flavivirus containing cell culture fluid. In another example, the second flavivirus with a high level of replication in a cell can be a flavivirus capable of high level replication in a cell as well as causing a persistent and non-pathogenic infection, e.g., allow infected cell culture to remain intact over a period of time (e.g., more than one day) and permit harvest of the cell culture supernatant fluid over a period of time (e.g., up to 4, 5, 6, or 7 days with daily harvest) to increase the volume of virus-containing cell culture fluid collected in every batch.

In certain embodiments, the second flavivirus is a flavivirus capable of high level replication in a cell and replicates without causing a significant elevation in the extracellular release of DNA, e.g., host DNA release into cell culture medium. In some embodiments, the second flavivirus is a flavivirus that does not cause an elevation, e.g., a measurable elevation in extracellular DNA release as compared to a mock infection when growing in a cell culture, and as measured at day 3 post-infection. In some other embodiments, the second flavivirus is a flavivirus that does not cause an elevation in extracellular DNA release at more than 5 ng/mL, 10 ng/mL, 15 ng/mL, 20 ng/mL, 30 ng/mL, or 100 ng/mL as compared to a mock infection when growing in a cell culture and measured at day 3 post-infection

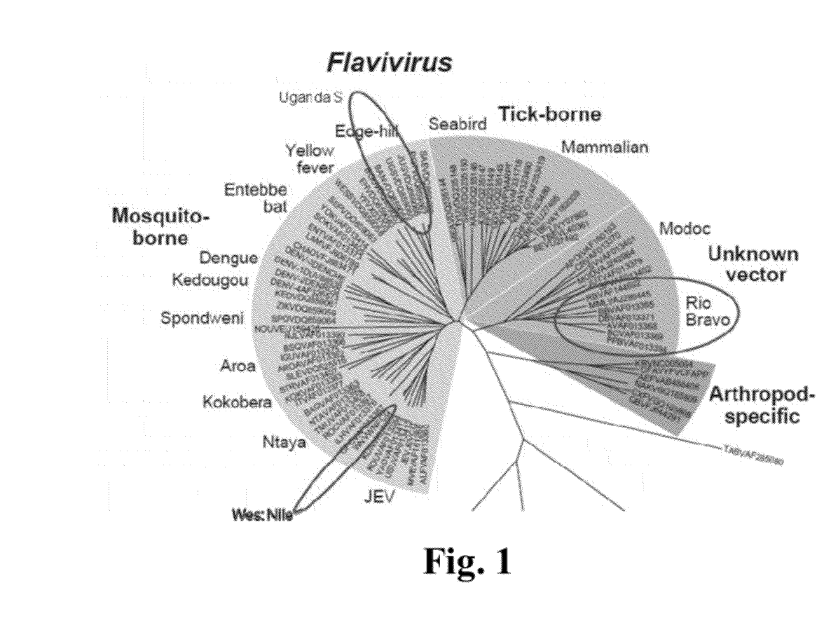

In certain embodiments, the second flavivirus is a flavivirus selected from the Rio Bravo taxonomic group. For example, the flavivirus of the Rio Bravo taxonomic group may be selected from the group consisting of the Rio Bravo (sensu stricto) virus, the Montana Myotis Leukoencephalitis virus, the Dakar Bat virus, the Phnom Penh Bat virus, the Carey Island virus, and the Bukalasa Bat virus. In an exemplary embodiment, the flavivirus of the Rio Bravo taxonomic group is the Rio Bravo virus (sensu stricto) virus. The phylogenetic relationship of the Rio Bravo taxonomic group of flaviviruses with the other members of the Flavivirus genus is illustrated in FIG. 1.

The invention is based, in part, on the discovery that members of the Rio Bravo taxonomic group are capable of replicating to very high titers (≧10⁸ PFUs/mL) in acceptable cell lines for manufacturing (e.g., Vero cells). Members of the Rio Bravo taxonomic group of viruses are not transmitted by arthropod vectors and are instead maintained in nature by direct transmission between a specific order of vertebrates (Chiroptera bats). The Rio Bravo virus has the advantage that they can be manipulated under Biological Safety Level 2 (BSL2) conditions, whereas the West Nile virus requires higher containment conditions (BSL3). In addition, Rio Bravo virus is capable of replicating at high level in cells without causing extensive cytopathic effects in cells and/or elevation in the extracellular release of host DNA so that multiple harvest of cell culture fluid can be made during manufacturing batches.

In certain other embodiments, the second flavivirus is a flavivirus selected from the Uganda S taxonomic group. For example, the flavivirus of the Uganda S taxonomic group may be selected from the group consisting of the Uganda S virus, the Banzi virus, and the Jugra virus. The phylogenetic relationship of the Uganda S taxonomic group of flaviviruses with the other members of the Flavivirus genus is also illustrated in FIG. 1. Similar to the Rio Bravo virus, the Uganda S taxonomic group of flaviviruses has the advantage that they can be manipulated under Biological Safety Level 2 (BSL2) conditions.

In still other embodiments, the second flavivirus is a flavivirus selected from the mosquito-associated flaviviruses. For example, the mosquito-associated flavivirus may be selected from the group consisting of the Kamiti River virus, the Culex flavivirus, the Aedes flavivirus, the Nakiwogo virus, the Quang Binh virus, and the Cell Fusing Agent virus. These mosquito-associated flaviviruses represent primitive insect viruses that are incapable of infecting mammalian cells. Examples of such viruses isolated from mosquitoes belonging to the genera Culex, Aedes, or Melanoconion mosquitoes include the Cell Fusing Agent virus (Stollar et al., 1975, Virology 64: 367-77), the Kamiti River virus (Crabtree et al., 2003, Arch Virol. 148: 1095-1118), the Culex flavivirus (Blivich et al., 2009, J Med. Entomol. 46: 934-41), the Aedes flavivirus (Hoshino et al., 2009, Virology 391: 119-29), the Nakiwogo virus (Cook et al., 2009, J Gen Virol 90: 2669-78), the Quang Binh virus (Crabtree et al., 2009, Arch Virol. 154: 857-60) and a variety of other agents (Tesh et al., 2009, Virology 386: 154-9). Selected mosquito-associated flaviviruses grow to high titers (>10¹⁰ PFU/mL) in cultures of insect cells, such as C6/36 Aedes albopictus mosquito cells, which can be employed for manufacturing vaccines. In certain embodiments, the chimeric flavivirus vectors comprising a backbone from a mosquito-associated flaviviruses may be capable of replicating in insect cells, but not capable of replicating in human cells. In addition to C6/36 Aedes albopictus mosquito cells, non-limiting examples of other insect cell lines for use in the present invention include u4.4 cells, High Five™ cells, Schneider's Drosophila cell line 2, Spodoptera frugiperda SF9 cells, Anopheles albimanus cells, Anopheles gambiae cells, Culex tarsalis cells, Phlebotomus papatasi cells, and C7-10 cells.

Because the mosquito-associated flaviviruses are incapable of replicating in mammalian cells or mammals, including humans, after injection, chimeric flaviviruses derived from chimeric flavivirus vectors comprising a backbone from a mosquito-associated flaviviruses may be used in a vaccine without prior inactivation. Further, as described herein, such chimeric flaviviruses derived from chimeric flavivirus vectors comprising a backbone from a mosquito-associated flaviviruses may be used in a vaccine without the addition of an adjuvant, because the viral RNA genome remains intact and acts as a self-adjuvant.

In still other embodiments, second flavivirus may be a flavivirus selected from the group consisting of the West Nile virus, the Rocio virus, the Ilheus virus, the Japanese encephalitis virus or the Murray Valley encephalitis virus.

In some embodiments, the chimeric flavivirus vector encodes a chimeric flavivirus capable of replicating in mammalian cells to at least about 10⁸ PFU/mL. In a further embodiment, the chimeric flavivirus vector encodes a chimeric flavivirus capable of replicating in mammalian cells to at least about 10⁹ PFU/mL.

As described above, in some embodiments, the chimeric flavivirus vectors of the invention comprise a backbone from a flavivirus with a high level of replication in the cell. In order to enhance the growth of selected viruses (e.g., a flavivirus with a high level of replication in the cell), serial passages may be employed in cell culture to “adapt” the virus to grow to even higher titer. This step may result in specific mutations or deletions in the virus genome. The adapted-mutated virus strain may be used to prepare an infectious clone, as described herein. Alternatively, if an infectious clone has already been prepared, the adaptation mutations can be inserted by site-directed mutagenesis, resulting in a virus that grows optimally in the selected cell culture.

After selection of the vector virus strain based on its growth characteristics, it is adapted by serial passage in selected cell type, and a stock of virus prepared by passage in the same cells. Another growth curve is performed to confirm that the virus yield is similar to that seen with the unadapted virus. The RNA of the adapted virus (e.g., Rio Bravo virus) is extracted and reverse transcribed to the complementary DNA sequence using reverse transcriptase for sequencing to determine which mutations have occurred that are associated with adaptation to the cell substrate.

In certain embodiments, the backbone may be selected from a flavivirus that has been adapted to a cell substrate. For example, the backbone may be selected from a flavivirus that has been adapted to Vero, Madin Darby Canine kidney (MDCK), A549, fetal rhesus lung (FRhL), MRC5, human embryonic kidney (HEK293), primary dog kidney, primary rabbit kidney, xenopus oocytes, or chick embryo cells. In some embodiments, the backbone is selected from a flavivirus adapted to a cell substrate such that an increase in yield of the virus is at least about 2.5-fold as compared to the flavivirus before adaptation.

In additional embodiments, the backbone may comprise at least one amino acid modification in a non-structural protein derived from a flavivirus with a high level of replication in the cell. In some embodiments, the non-structural protein is selected from the group consisting of NS1, NS2A, NS2B, NS3, NS4A, NS4B, and NS5. In additional embodiments, the backbone may comprise at least one amino acid modification in at least two non-structural proteins selected from the group consisting of NS1, NS2A, NS2B, NS3, NS4A, NS4B, and NS5. In certain specific embodiments, the non-structural protein is NS1. For example, the NS1 protein may comprise a substitution at an amino acid position corresponding to the proline 315 residue of the Rio Bravo virus NS1 (SEQ ID NO: 1). In further specific embodiments, the proline 315 residue of the NS1 protein is replaced with a serine residue. In certain specific embodiments, the non-structural protein is NS3. For example, the NS3 protein may comprise a substitution at an amino acid position corresponding to the isoleucine 555 residue of the Rio Bravo virus NS3 (SEQ ID NO: 2). In further specific embodiments, the isoleucine 555 residue of the NS3 protein is replaced with a threonine residue.

In some embodiments, the chimeric flavivirus vector may comprise at least one nucleotide deletion in the 3′ non-coding region (NCR). For example, the 3′ non-coding region may comprise a nucleotide deletion at a nucleotide corresponding to the thymine at position 10692 of the Rio Bravo virus genome. In additional embodiments, the chimeric flavivirus vector may comprise at least one nucleotide deletion in the 5′ non-coding region (NCR).

In one embodiment, the chimeric flavivirus vector may further comprise a signal sequence at the 3′ end of the capsid gene. In another embodiment, the chimeric flavivirus vector may be within a plasmid comprising at least one cytomegalovirus (CMV) promoter, e.g., operably linked to the chimeric flavivirus vector. In yet another embodiment, the chimeric flavivirus vector may be within a plasmid expressing at least one hepatitis δ virus ribozyme. In yet another embodiment, the chimeric flavivirus vector may be within a plasmid comprising at least one SV40 polyadenylation site. In yet another embodiment, the chimeric flavivirus vector may comprise one or more intron sequences. For example, in one embodiment, the chimeric flavivirus vector may comprise an intron at the junction of the envelope (E) and the non-structural gene 1 (NS1). In another embodiment, the chimeric flavivirus vector may comprise an intron immediately after a nucleotide corresponding to position 9742 of the Rio Bravo virus genome.

### Methods of Culturing Chimeric Flavivirus Vectors to Produce Chimeric Flaviviruses

In another aspect, the invention provides chimeric flaviviruses encoded by a chimeric flavivirus vector described herein. In one embodiment, the chimeric flavivirus vector comprises a sequence encoding a structural protein from a first flavivirus with a low level of replication in a cell and a backbone from a second flavivirus with a high level of replication in the cell. In another embodiment, the chimeric flavivirus vector encodes a chimeric flavivirus, wherein the chimeric flavivirus comprises a structural protein from a first flavivirus with a low level of replication in a cell and 1) a capsid protein from a second flavivirus with a high level of replication in the cell, 2) an RNA genome comprising a sequence encoding one or more or all of the non-structural proteins, e.g., NS1, NS2A, NS2B, NS3, NS4A, NS4B, and NS5 from a second flavivirus with a high level of replication in the cell, or 3) an RNA genome with both 3′ and 5′ termini of the genome from a second flavivirus with a high level of replication in the cell, or a combination thereof.

In another aspect, the invention provides methods of culturing one or more chimeric flavivirus vectors to produce chimeric flavivirus, e.g., growing chimeric flavivirus in cell cultures. In one embodiment, the chimeric flavivirus vector comprises a sequence encoding a structural protein from a first flavivirus with a low level of replication in a cell and a backbone from a second flavivirus with a high level of replication in the cell. In another embodiment, the chimeric flavivirus vector encodes a chimeric flavivirus, wherein the chimeric flavivirus comprises a structural protein from a first flavivirus with a low level of replication in a cell and 1) a capsid protein from a second flavivirus with a high level of replication in the cell, 2) an RNA genome comprising a sequence encoding one or more or all of the non-structural proteins, e.g., NS1, NS2A, NS2B, NS3, NS4A, NS4B, and NS5 from a second flavivirus with a high level of replication in the cell, or 3) an RNA genome with both 3′ and 5′ termini of the genome from a second flavivirus with a high level of replication in the cell, or a combination thereof.

In a method to culture one or more chimeric flavivirus vectors to produce a chimeric flavivirus, the one or more chimeric flavivirus vectors may be used to infect an appropriate cell line. A cell culture strain for production is selected based on demonstrating maximum growth of both the second flavivirus with a high level of replication in the cell and the first flavivirus strain with a low level of replication in a cell which is donating a structural protein (e.g., an envelope and/or a pre-membrane protein).

The cells used for growth of the chimeric flavivirus can be one of a number of suitable cell cultures for vaccine development, including primary chick embryo cells, primary duck embryo cells, primary rabbit kidney, primary dog kidney, diploid continuous embryonic avian cell lines, mammalian diploid cells such as fetal rhesus lung (FRhL) or MRC5, or heteroploid cells such as Vero cells, PerC6, 293T, 293S, Madin Darby Canine Kidney (MDCK), human embryonic kidney (HEK293), xenopus oocytes, or A549 cells. In certain exemplary embodiments, the cells are Vero cells.

In the case of chimeric flavivirus vectors comprising a backbone from a mosquito-associated flavivirus, these chimeric flavivirus vectors may be capable of replicating in insect cells, but not capable of replicating in human cells. Non-limiting examples of insect cell lines for use in culturing chimeric flavivirus vectors comprising a backbone from a mosquito-associated flavivirus include C6/36 Aedes albopictus mosquito cells, u4.4 cells, High Five™ cells, Schneider's Drosophila cell line 2, Spodoptera frugiperda SF9 cells, Anopheles albimanus cells, Anopheles gambiae cells, Culex tarsalis cells, Phlebotomus papatasi cells, and C7-10 cells.

After the chimeric flavivirus vector infects an appropriate cell line, the cell line may be cultured to produce a cell culture supernatant comprising the chimeric flavivirus, e.g., the virus is released into the cell culture supernatant from which it may be harvested. Preferably, growth of the chimeric flavivirus in cell culture is performed without the addition of bovine serum, porcine trypsin and other animal derived products which can be the source of adventitious viruses. In certain embodiments, the cell culture may be grown in suspension, on microcarrier beads, or on the surface of tissue culture flasks, cell factories or roller bottles. Following growth of the chimeric flavivirus in cell culture, the chimeric flavivirus may be harvested from the cell culture supernatant.

In some embodiments, the chimeric flavivirus vector encodes a chimeric flavivirus capable of replicating in mammalian cells to at least about 10⁸ PFU/mL. In a further embodiment, the chimeric flavivirus vector encodes a chimeric flavivirus capable of replicating in mammalian cells to at least about 10⁹ PFU/mL. In additional embodiments, the chimeric flavivirus vector encodes a chimeric flavivirus capable of replicating in insect cells, but not capable of replicating in human cells.

### Methods of Inactivating Chimeric Flaviviruses to Produce Inactivated Chimeric Flaviviruses

In yet another aspect, the invention provides an inactivated chimeric flavivirus encoded by a chimeric flavivirus vector described herein. In one embodiment, the chimeric flavivirus comprises a structural protein from a first flavivirus with a low level of replication in a cell, e.g., an envelope protein and optionally a membrane protein and 1) a capsid protein from a second flavivirus with a high level of replication in the cell, 2) an RNA genome comprising a sequence encoding one or more or all of the non-structural proteins, e.g., NS1, NS2A, NS2B, NS3, NS4A, NS4B, and NS5 from a second flavivirus with a high level of replication in the cell, or 3) an RNA genome with both 3′ and 5′ termini of the genome from a second flavivirus with a high level of replication in the cell, or a combination thereof. The preparation of an inactivated chimeric flavivirus according to the present invention can be obtained by routine methods well known to the person skilled in the art. General methods for the inactivation of viral pathogens are described in US 2006/0270017, which is hereby incorporated by reference in its entirety. Downstream manufacturing steps used to purify and inactivate whole virion vaccines against other Flaviviruses (e.g., Japanese encephalitis, tick-borne encephalitis, and West Nile virus) have been described previously. See, e.g., WO/2006/122964. See also Srivastava et al., 2001, Vaccine 19: 4557-65.

In certain embodiments, the chimeric flavivirus encoded by a chimeric flavivirus vector may be inactivated with a method selected from the group consisting of chemical inactivation, high pressure inactivation, ultraviolet radiation, and gamma radiation. In an exemplary embodiment, the chimeric flavivirus is inactivated using chemical inactivation. For example, the method of chemical inactivation may comprise exposure of the flavivirus to one or more agents selected from the group consisting of β-propiolactone, formalin, aziridines, hydrogen peroxide, organic solvents, and ascorbic acid. In an exemplary embodiment, the chimeric flavivirus is inactivated may be inactivated by exposure to β-propiolactone.

After the chimeric flavivirus has been inactivated, the inactivated chimeric flavivirus may then be purified for use in various applications, including vaccine and other pharmaceutical compositions. In an exemplary embodiment, the method of inactivation further comprises a step of purifying the inactivated chimeric flavivirus in the sample to pharmaceutical purity and formulating the purified chimeric flavivirus into a pharmaceutical composition for use as a vaccine. Purification may be accomplished by any means known in the art, including, but not limited to, filtration or diafiltration, chromatography (e.g., size exclusion, ion exchange, immunoaffinity, and the like) or centrifugation. Alternatively, the chimeric flavivirus may be purified prior to inactivation by the methods of the invention.

### Vaccines Comprising an Chimeric Flavivirus

In yet another aspect, the invention provides a vaccine comprising a chimeric flavivirus described herein. In one embodiment, the vaccine comprises an inactivated chimeric flavivirus encoded by a chimeric flavivirus vector described herein. In another embodiment, the vaccine comprises an inactivated chimeric flavivirus, wherein the inactivated chimeric flavivirus is derived from a chimeric flavivirus comprising a structural protein, e.g., an envelope (E) and optionally a membrane protein from a first flavivirus with a low level of replication in a cell and 1) a capsid protein from a second flavivirus with a high level of replication in the cell, 2) an RNA genome comprising a sequence encoding one or more or all of the non-structural proteins, e.g., NS1, NS2A, NS2B, NS3, NS4A, NS4B, and NS5 from a second flavivirus with a high level of replication in the cell, or 3) an RNA genome with both 3′ and 5′ termini of the genome from a second flavivirus with a high level of replication in the cell, or a combination thereof.

In some embodiments, the inactivated chimeric flavivirus is derived from a chimeric flavivirus comprising a structural protein, e.g., an envelope (E) and optionally a membrane protein from a first flavivirus with a low level of replication in a cell, wherein the first flavivirus with a low level of replication in a cell is a dengue virus. In a further embodiment, the dengue virus may be selected from the group consisting of dengue type 1 (DEN1) virus, dengue type 2 (DEN2) virus, dengue type 3 (DEN3) virus, and dengue type 4 (DEN4) virus.

In certain other embodiments, the inactivated chimeric flavivirus is derived from a chimeric flavivirus comprising a structural protein, e.g., an envelope (E) and optionally a membrane protein from a first flavivirus with a low level of replication in a cell, wherein the first flavivirus with a low level of replication in a cell is a yellow fever virus.

In some embodiments, the inactivated chimeric flavivirus is derived from a chimeric flavivirus comprising 1) a capsid protein from a second flavivirus with a high level of replication in the cell, 2) an RNA genome comprising a sequence encoding one or more or all of the non-structural proteins, e.g., NS1, NS2A, NS2B, NS3, NS4A, NS4B, and NS5 from a second flavivirus with a high level of replication in the cell, or 3) an RNA genome with both 3′ and 5′ termini of the genome from a second flavivirus with a high level of replication in the cell, or a combination thereof, wherein the second flavivirus is a flavivirus capable of high level replication in a cell and replicates without causing extensive cytopathic effects (CPE) in the cell, e.g., without causing substantial degenerative changes in the cell. For example, the second flavivirus with a high level of replication in a cell can be a flavivirus capable of high level replication in a cell as well as allowing more than one harvest of cell culture fluid, e.g., multiple harvest of flavivirus containing cell culture fluid. In another example, the second flavivirus with a high level of replication in a cell can be a flavivirus capable of high level replication in a cell as well as causing a persistent and non-pathogenic infection, e.g., allow infected cell culture to remain intact over a period of time (e.g., more than one day) and permit harvest of the cell culture supernatant fluid over a period of time (e.g., up to 4, 5, 6, or 7 days with daily harvest) to increase the volume of virus-containing cell culture fluid collected in every batch.

In some embodiments, the inactivated chimeric flavivirus is derived from a chimeric flavivirus comprising 1) a capsid protein from a second flavivirus with a high level of replication in the cell, 2) an RNA genome comprising a sequence encoding one or more or all of the non-structural proteins, e.g., NS1, NS2A, NS2B, NS3, NS4A, NS4B, and NS5 from a second flavivirus with a high level of replication in the cell, or 3) an RNA genome with both 3′ and 5′ termini of the genome from a second flavivirus with a high level of replication in the cell, or a combination thereof, wherein the second flavivirus is a flavivirus capable of high level replication in a cell and replicates without causing a significant elevation in the extracellular release of DNA, e.g., host DNA release into cell culture medium. In some embodiments, the second flavivirus is a flavivirus that does not cause an elevation, e.g., a measurable elevation in extracellular DNA release as compared to a mock infection when growing in a cell culture, and as measured at day 3 post-infection. In some other embodiments, the second flavivirus is a flavivirus that does not cause an elevation in extracellular DNA release at more than 5 ng/mL, 10 ng/mL, 15 ng/mL, 20 ng/mL, 30 ng/mL, or 100 ng/mL as compared to a mock infection when growing in a cell culture and measured at day 3 post-infection

In some embodiments, the inactivated chimeric flavivirus is derived from a chimeric flavivirus comprising 1) a capsid protein from a second flavivirus with a high level of replication in the cell, 2) an RNA genome comprising a sequence encoding one or more or all of the non-structural proteins, e.g., NS1, NS2A, NS2B, NS3, NS4A, NS4B, and NS5 from a second flavivirus with a high level of replication in the cell, or 3) an RNA genome with both 3′ and 5′ termini of the genome from a second flavivirus with a high level of replication in the cell, or a combination thereof, wherein the second flavivirus with a high level of replication in the cell is a flavivirus selected from the Rio Bravo taxonomic group. For example, the flavivirus of the Rio Bravo taxonomic group may be selected from the group consisting of the Rio Bravo (sensu stricto) virus, the Montana Myotis Leukoencephalitis virus, the Dakar Bat virus, the Phnom Penh Bat virus, the Carey Island virus, and the Bukalasa Bat virus.

In certain other embodiments, the inactivated chimeric flavivirus is derived from a chimeric flavivirus comprising 1) a capsid protein from a second flavivirus with a high level of replication in the cell, 2) an RNA genome comprising a sequence encoding one or more or all of the non-structural proteins, e.g., NS1, NS2A, NS2B, NS3, NS4A, NS4B, and NS5 from a second flavivirus with a high level of replication in the cell, or 3) an RNA genome with both 3′ and 5′ termini of the genome from a second flavivirus with a high level of replication in the cell, or a combination thereof, wherein the second flavivirus with a high level of replication in the cell is a flavivirus selected from the Uganda S taxonomic group. For example, the flavivirus of the Uganda S taxonomic group may be selected from the group consisting of the Uganda S virus, the Banzi virus, and the Jugra virus.

In yet other embodiments, the inactivated chimeric flavivirus is derived from a chimeric flavivirus comprising 1) a capsid protein from a second flavivirus with a high level of replication in the cell, 2) an RNA genome comprising a sequence encoding one or more or all of the non-structural proteins, e.g., NS1, NS2A, NS2B, NS3, NS4A, NS4B, and NS5 from a second flavivirus with a high level of replication in the cell, or 3) an RNA genome with both 3′ and 5′ termini of the genome from a second flavivirus with a high level of replication in the cell, or a combination thereof, wherein the second flavivirus with a high level of replication in the cell is a flavivirus selected from the mosquito-associated flaviviruses. For example, the mosquito-associated flavivirus may be selected from the group consisting of the Kamiti River virus, the Culex flavivirus, the Aedes flavivirus, the Nakiwogo virus, the Quang Binh virus, and the Cell Fusing Agent virus. In still yet other embodiments, the inactivated chimeric flavivirus is derived from a chimeric flavivirus comprising 1) a capsid protein from a second flavivirus with a high level of replication in the cell, 2) an RNA genome comprising a sequence encoding one or more or all of the non-structural proteins, e.g., NS1, NS2A, NS2B, NS3, NS4A, NS4B, and NS5 from a second flavivirus with a high level of replication in the cell, or 3) an RNA genome with both 3′ and 5′ termini of the genome from a second flavivirus with a high level of replication in the cell, or a combination thereof, wherein the second flavivirus with a high level of replication in the cell may be a flavivirus selected from the group consisting of the West Nile virus, the Rocio virus, the Ilheus virus, the Japanese encephalitis virus or the Murray Valley encephalitis virus.

In alternative embodiments, the vaccine comprises a live, chimeric flavivirus, wherein the live chimeric flavivirus comprises a structural protein from a first flavivirus with a low level of replication in a cell and 1) a capsid protein from a second flavivirus with a high level of replication in the cell, 2) an RNA genome comprising a sequence encoding one or more or all of the non-structural proteins, e.g., NS1, NS2A, NS2B, NS3, NS4A, NS4B, and NS5 from a second flavivirus with a high level of replication in the cell, or 3) an RNA genome with both 3′ and 5′ termini of the genome from a second flavivirus with a high level of replication in the cell, or a combination thereof. In an exemplary embodiment, the live, chimeric flavivirus comprises a structural protein from a first flavivirus with a low level of replication in a cell and 1) a capsid protein from a mosquito-associated flavivirus, 2) an RNA genome comprising a sequence encoding one or more or all of the non-structural proteins, e.g., NS1, NS2A, NS2B, NS3, NS4A, NS4B, and NS5 from a mosquito-associated flavivirus, or 3) an RNA genome with both 3′ and 5′ termini of the genome from a mosquito-associated flavivirus, or a combination thereof. In certain embodiments, the mosquito-associated flavivirus may be selected from the group consisting of the Kamiti River virus, the Culex flavivirus, the Aedes flavivirus, the Nakiwogo virus, the Quang Binh virus, and the Cell Fusing Agent virus. In additional embodiments, the vaccine comprising a live, chimeric flavivirus may be administered to a subject in need thereof in the absence of adjuvant.

In still yet another aspect, the invention provides a vaccine comprising a chimeric flavivirus vector described herein. In some embodiments, the invention provides a DNA vaccine comprising a chimeric flavivirus vector described herein. In some other embodiments, the invention provides a DNA vaccine comprising a chimeric flavivirus vector described herein and suitable for delivery by gene gun or any other DNA vaccine delivery methods.

As used herein, the term “vaccine” refers to a composition that comprises one or more chimeric flavivirus vectors and/or chimeric flaviviruses of the invention. In certain embodiments, the chimeric flavivirus of the vaccine is an inactivated chimeric flavivirus. In certain other embodiments, the chimeric flavivirus of the vaccine is a live, chimeric flavivirus. In certain exemplary embodiments, the vaccine may comprise a mixture of a first, second, third, and/or fourth chimeric flavivirus vectors wherein the envelope protein in the first, second, third, and/or fourth chimeric flavivirus vector is derived from a dengue type 1 (DEN1) virus, dengue type 2 (DEN2) virus, dengue type 3 (DEN3) virus, and dengue type 4 (DEN 4) virus, respectively. In certain other exemplary embodiments, the vaccine may comprise a mixture of a first, second, third, and/or fourth chimeric flaviviruses encoded by a first, second, third, and/or fourth chimeric flavivirus vector, wherein the envelope protein in the first, second, third, and/or fourth is derived from a dengue type 1 (DEN1) virus, dengue type 2 (DEN2) virus, dengue type 3 (DEN3) virus, and dengue type 4 (DEN 4) virus, respectively. In one embodiment, one or more of the chimeric flaviviruses of said mixture has been inactivated. In another embodiment, all chimeric flaviviruses of said mixture have been inactivated. In yet another embodiment, one or more of the chimeric flaviviruses of said mixture is a live, chimeric flavivirus. In yet another embodiment, all chimeric flaviviruses of said mixture are live, chimeric flaviviruses.

In certain embodiments, the vaccine further comprises an adjuvant. Examples of such adjuvants include, but are not limited to, salts, such as calcium phosphate, aluminum phosphate, calcium hydroxide and aluminum hydroxide; natural polymers such as algal glucans (e.g., beta glucans), chitosan or crystallized inulin; synthetic polymers such as poly-lactides, poly-glycolides, poly lacitide-co-glycolides or methylacrylate polymers; oil-in-water emulsions such as MF59; water-in-oil emulsions, micelle-forming cationic or non-ionic block copolymers or surfactants such as Pluronics, L121, 122 or 123, Tween 80, or NP-40; fatty acid, lipid (e.g., lipid A or monophosphoryl lipid A) or lipid and protein based vesicles such as liposomes, proteoliposomes, ISCOM, ISCOMATRIX, and cochleate structures; surfactant stabilized emulsions composed of synthetic or natural oils and aqueous solutions, immunostimulatory oligonucleotides (e.g., CpGs), and poly I:C.

In certain embodiments, a vaccine of the invention, upon administration to a subject, is capable of stimulating an immune response (e.g., a humoral immune response, cellular immune response, or both) in the subject. In certain embodiments, the immune response includes a measurable response (e.g., a measurable humoral or cellular immune response, or combination thereof) to an epitope encoded by a structural protein (e.g., a dengue virus or yellow fever virus envelope and/or a membrane protein) inserted or integrated into a chimeric flavivirus of the vaccine. In certain embodiments, a vaccine of the invention is capable of providing protection against dengue. In certain other embodiments, a vaccine of the invention is capable of providing protection against yellow fever. For example, in certain embodiments, the vaccine is capable of stimulating an immune response against one or more antigens (e.g., encoded by a dengue virus or yellow fever virus structural protein) such that, upon later encountering such an antigen, the subject receiving the vaccine has an immune response that is stronger than it would have been if the vaccine had not been administered previously. In other embodiments, a vaccine of the invention is capable of ameliorating a dengue virus infection and/or reducing at least one symptom of dengue virus infection. In yet other embodiments, a vaccine of the invention is capable of ameliorating a yellow fever virus infection and/or reducing at least one symptom of yellow fever virus infection. For instance, in one embodiment, the vaccine of the invention induces a therapeutic immune response against one or more antigens (e.g., encoded by a dengue virus or yellow fever virus structural protein) such that symptoms and/or complications of an infection in a subject suffering from such an infection.

The chimeric flavivirus vectors and/or chimeric flaviviruses used for the vaccines can be prepared and formulated for administration to a mammal in accordance with techniques well known in the art. Formulations for oral administration can consist of capsules or tablets containing a predetermined amount of a chimeric flavivirus of the invention; liquid solutions, such as an effective amount of the pharmaceutical dissolved in ingestible diluents, such as water, saline, orange juice, and the like; suspensions in an appropriate liquid; and suitable emulsions.

The chimeric flavivirus vectors and/or chimeric flaviviruses of the invention can, for example, be formulated as enteric coated capsules for oral administration, as previously described, in order to bypass the upper respiratory tract and allow viral replication in the gut. See, e.g., Tacket et al., 1992, Vaccine 10: 673-6; Horwitz, in Fields et al., eds., 1996, Fields Virology, third edition, Vol 2: 2149-71; Takafuji et al., 1979, J. Infec. Dis. 140: 48-53; and Top et al., 1971, J. Infec. Dis. 124: 155-60. Alternatively, the chimeric flaviviruses can be formulated in conventional solutions, such as sterile saline, and can incorporate one or more pharmaceutically acceptable carriers or excipients. The pharmaceutical composition can further comprise other active agents.

In certain embodiments, formulations of the invention comprise a buffered solution comprising one or more chimeric flaviviruses of the invention in a pharmaceutically acceptable carrier. A variety of carriers can be used, such as buffered saline, water and the like. Such solutions are generally sterile and free of undesirable matter. These compositions can be sterilized by conventional, well known sterilization techniques, or can be sterile filtered. The compositions may contain pharmaceutically acceptable auxiliary substances as required to approximate physiological conditions such as pH adjusting and buffering agents, tonicity adjusting agents and the like, for example, sodium acetate, sodium chloride, potassium chloride, calcium chloride, sodium lactate and the like.

Pharmaceutically acceptable carriers can contain a physiologically acceptable compound that acts, e.g., to stabilize the composition or to increase or decrease the absorption of the virus and/or pharmaceutical composition. Physiologically acceptable compounds can include, for example, carbohydrates, such as glucose, sucrose, or dextrans, antioxidants, such as ascorbic acid or glutathione, chelating agents, low molecular weight proteins, compositions that reduce the clearance or hydrolysis of any co-administered agents, or excipient, or other stabilizers and/or buffers. Detergents can also be used to stabilize the composition or to increase or decrease absorption. Detergents may be used to ‘split’ the virus by disrupting the lipid-containing envelope of the virion. One skilled in the art will appreciate that the choice of a pharmaceutically acceptable carrier, including a physiologically acceptable compound depends, e.g., on the route of administration of the adenoviral preparation and on the particular physio-chemical characteristics of any co-administered agent.

The chimeric flavivirus vectors and/or chimeric flaviviruses of the invention can also be administered in a lipid formulation, more particularly either complexed with liposomes or to lipid/nucleic acid complexes or encapsulated in liposomes. The chimeric flavivirus vectors and/or chimeric flaviviruses of the current invention, alone or in combination with other suitable components, can also be made into aerosol formulations to be administered via inhalation. The vaccines can also be formulated for administration via the nasal passages. Formulations suitable for nasal administration, wherein the carrier is a solid, include a coarse powder having a particle size, for example, in the range of about 10 to about 500 microns which is administered in the manner in which snuff is taken, i.e., by rapid inhalation through the nasal passage from a container of the powder held close up to the nose. Suitable formulations wherein the carrier is a liquid for administration as, for example, nasal spray, nasal drops, or by aerosol administration by nebulizer, include aqueous or oily solutions of the active ingredient. In some embodiments, the chimeric flaviviruses of the invention can be formulated as suppositories, for example, for rectal or vaginal administration.

In the case of DNA vaccines, e.g., vaccines comprising a chimeric flavivirus vector of the invention, these DNA vaccines can be delivered by different routes of administration. For example, the DNA vaccines, e.g., vaccines comprising a chimeric flavivirus vector described herein, may be combined with a physiologically acceptable carrier prior to in vivo administration. The DNA vaccine may be administered by routes including, but not limited to, inhalation, intradermal injection, intramuscular injection, intravenous injection, intraperitoneal injection, subcutaneous injection, application to mucosal surfaces (e.g., application of DNA drops to the nares or trachea), intraocular administration, or particle bombardment of the epidermis using a gene gun. In some embodiments, DNA vaccines can be injected in saline solutions into muscle or skin using a syringe and needle. See, e.g., US 2002/0025939. DNA vaccines can also be administered by coating the nucleic acid onto microscopic gold beads and then using a gene gun to deliver the beads into cells. The saline injections deliver the nucleic acid into extracellular spaces, whereas gene guns deliver nucleic acid coated gold beads directly into cells.

Vaccines comprising a chimeric flavivirus vectors and/or chimeric flavivirus of the invention can have a unit dosage comprising between about 5 μg to about 100 μg (e.g., about 5 μg to about 15 μg, about 15 μg to about 25 μg, about 25 μg to about 35 μg, about 35 μg to about 45 μg, about 45 μg to about 55 μg, about 55 μg to about 65 μg, about 65 μg to about 75 μg, about 75 μg to about 85 μg, about 85 μg to about 95 μg, and about 95 μg to about 100 μg) of the chimeric flavivirus in a single dose. The dosages can vary based on the route of administration. For instance, vaccines formulated for sublingual or intranasal administration may contain a lower dosage of chimeric flavivirus per single dose than vaccines formulated for alternative routes of administration, e.g., the oral routes of administration. One of skill in the art can determine the appropriate dosage for a particular patient depending on the type of infection, and the route of administration to be used without undue experimentation.

### Methods of Inducing an Immune Response

In yet another aspect, the invention provides a method for inducing an immune response in a subject, wherein the method comprises administration of a chimeric flavivirus vectors described herein and/or chimeric flavivirus encoded by a chimeric flavivirus vector described herein. In one embodiment, the administration includes a single dose administration. In another embodiment, the administration includes no more than two, three, or four doses of administration. In some embodiments, the chimeric flavivirus is an inactivated chimeric flavivirus. In some embodiments, the chimeric flavivirus comprises a structural protein, e.g., an envelope (E) and optionally a membrane protein from a first flavivirus with a low level of replication in a cell and 1) a capsid protein from a second flavivirus with a high level of replication in the cell, 2) an RNA genome comprising a sequence encoding one or more or all of the non-structural proteins, e.g., NS1, NS2A, NS2B, NS3, NS4A, NS4B, and NS5 from a second flavivirus with a high level of replication in the cell, or 3) an RNA genome with both 3′ and 5′ termini of the genome from a second flavivirus with a high level of replication in the cell, or a combination thereof. In a further embodiment, the inactivated chimeric flavivirus is administered in conjunction with an adjuvant.

In an alternative aspect, the invention provides a method for inducing an immune response in a subject, wherein the method comprises administration of a live, chimeric flavivirus encoded by a chimeric flavivirus vector described herein. In an exemplary embodiment, the live, chimeric flavivirus comprises a structural protein from a first flavivirus with a low level of replication in a cell and 1) a capsid protein from a mosquito-associated flavivirus, 2) an RNA genome comprising a sequence encoding one or more or all of the non-structural proteins, e.g., NS1, NS2A, NS2B, NS3, NS4A, NS4B, and NS5 from a mosquito-associated flavivirus, or 3) an RNA genome with both 3′ and 5′ termini of the genome from a mosquito-associated flavivirus, or a combination thereof. In certain embodiments, the mosquito-associated flavivirus may be selected from the group consisting of the Kamiti River virus, the Culex flavivirus, the Aedes flavivirus, the Nakiwogo virus, the Quang Binh virus, and the Cell Fusing Agent virus. In additional embodiments, the vaccine comprising a live, chimeric flavivirus may be administered to a subject in need thereof in the absence of adjuvant.

In one embodiment, the invention provides methods of inducing an immune response to dengue virus in a subject comprising administering to the subject a vaccine of the invention. In certain exemplary embodiments, the vaccine may comprise a mixture of a first, second, third, and/or fourth chimeric flavivirus vectors wherein the envelope protein in the first, second, third, and/or fourth is derived from a dengue type 1 (DEN1) virus, dengue type 2 (DEN2) virus, dengue type 3 (DEN3) virus, and dengue type 4 (DEN 4) virus, respectively. In certain exemplary embodiments, the vaccine may comprise a mixture of chimeric flaviviruses encoded by a first, second, third, and/or fourth chimeric flavivirus vector, wherein the envelope protein in the first, second, third, and/or fourth is derived from a dengue type 1 (DEN1) virus, dengue type 2 (DEN2) virus, dengue type 3 (DEN3) virus, and dengue type 4 (DEN 4) virus, respectively. In one embodiment, one or more of the chimeric flaviviruses of said mixture has been inactivated. In another embodiment, all chimeric flaviviruses of said mixture have been inactivated. In yet another embodiment, one or more of the chimeric flaviviruses of said mixture is a live, chimeric flavivirus. In yet another embodiment, all chimeric flaviviruses of said mixture are live, chimeric flaviviruses.

In another embodiment, the invention provides methods of inducing an immune response to yellow fever virus in a subject comprising administering to the subject a vaccine of the invention. In certain exemplary embodiments, the vaccine may comprise a chimeric flavivirus, wherein the chimeric flavivirus is derived from a chimeric flavivirus vector comprising a sequence encoding a structural protein from a yellow fever virus and a backbone from a second flavivirus with a high level of replication in the cell. In certain exemplary embodiments, the vaccine may comprise a chimeric flavivirus vector, comprising a sequence encoding a structural protein from a yellow fever virus and a backbone from a second flavivirus with a high level of replication in the cell.

In yet another aspect, the invention provides a method of vaccinating a subject against an infectious pathogen comprising administering a sufficient amount of a vaccine of the invention to a subject at risk for being infected by an infectious pathogen. In one embodiment, the administration includes a single dose administration. In another embodiment, the administration includes no more than two, three, or four doses of administration. In some embodiments, the vaccine comprises the chimeric flavivirus vector of the invention. In some other embodiment, the vaccine comprises an inactivated chimeric flavivirus. In yet some embodiments, the chimeric flavivirus comprises a structural protein, e.g., an envelope (E) and optionally a membrane protein from a first flavivirus with a low level of replication in a cell and 1) a capsid protein from a second flavivirus with a high level of replication in the cell, 2) an RNA genome comprising a sequence encoding one or more or all of the non-structural proteins, e.g., NS1, NS2A, NS2B, NS3, NS4A, NS4B, and NS5 from a second flavivirus with a high level of replication in the cell, or 3) an RNA genome with both 3′ and 5′ termini of the genome from a second flavivirus with a high level of replication in the cell, or a combination thereof.

In a further embodiment, the inactivated chimeric flavivirus is administered in conjunction with an adjuvant. In an alternative embodiment, the vaccine comprises a live, chimeric flavivirus. In an exemplary embodiment, the vaccine comprises a live, chimeric flavivirus, wherein the live chimeric flavivirus comprises a structural protein from a first flavivirus with a low level of replication in a cell and 1) a capsid protein from a mosquito-associated flavivirus, 2) an RNA genome comprising a sequence encoding one or more or all of the non-structural proteins, e.g., NS1, NS2A, NS2B, NS3, NS4A, NS4B, and NS5 from a mosquito-associated flavivirus, or 3) an RNA genome with both 3′ and 5′ termini of the genome from a mosquito-associated flavivirus, or a combination thereof. In certain embodiments, the mosquito-associated flavivirus may be selected from the group consisting of the Kamiti River virus, the Culex flavivirus, the Aedes flavivirus, the Nakiwogo virus, the Quang Binh virus, and the Cell Fusing Agent virus. In additional embodiments, the vaccine comprising a live chimeric flavivirus may be administered to a subject in need thereof in the absence of adjuvant.

In one embodiment, the invention provides a method of vaccinating a subject against a dengue virus infection comprising administering to the subject a vaccine of the invention. In certain exemplary embodiments, the vaccine may comprise a mixture of chimeric flaviviruses encoded by a first, second, third, and/or fourth chimeric flavivirus vector, wherein the envelope protein in the first, second, third, and/or fourth is derived from a dengue type 1 (DEN1) virus, dengue type 2 (DEN2) virus, dengue type 3 (DEN3) virus, and dengue type 4 (DEN 4) virus, respectively. In one embodiment, one or more of the chimeric flaviviruses of said mixture has been inactivated. In another embodiment, all chimeric flaviviruses of said mixture have been inactivated. In yet another embodiment, one or more of the chimeric flaviviruses of said mixture is a live chimeric flavivirus. In yet another embodiment, all chimeric flaviviruses of said mixture are live chimeric flaviviruses.

In another embodiment, the invention provides a method of vaccinating a subject against a yellow fever virus infection comprising administering to the subject a vaccine of the invention. In certain exemplary embodiments, the vaccine may comprise a chimeric flavivirus, wherein the chimeric flavivirus is derived from a chimeric flavivirus vector comprising a sequence encoding a structural protein from a yellow fever virus and a backbone from a second flavivirus with a high level of replication in the cell.

In another embodiment, the subject has an infection induced by the infectious pathogen (e.g., dengue virus or yellow fever virus). Thus, for instance, in one embodiment, the present invention provides a method of inducing a therapeutic immune response in a subject experiencing an infection induced by an infectious pathogen (e.g., dengue virus or yellow fever virus). In some embodiments, one or more symptoms or complications of the infection (e.g., dengue virus infection or yellow fever virus infection) is reduced or alleviated in the subject following administration of the vaccine. The vaccines of the invention can be used to vaccinate human or veterinary subjects.

The vaccines of the invention can be administered alone, or can be co-administered or sequentially administered with other immunological, antigenic, vaccine, or therapeutic compositions. Such compositions can include other agents to potentiate or broaden the immune response, e.g., IL-2 or other cytokines which can be administered at specified intervals of time, or continuously administered (see, e.g., Smith et al., 1997, N Engl J Med 336(17): 1260-1; and Smith et al., 1997, Cancer J Sci Am. 3 Suppl 1: S137-40). The vaccines of the invention can also be administered in conjunction with other vaccines, vectors, or viruses. For example, a chimeric flavivirus of the invention can be administered either before or after administration of another vaccine, e.g., another vaccine to an unrelated agents including without any limitation hepatitis A, hepatitis B, or typhoid vaccines or a combination thereof.

The chimeric flavivirus vectors and/or chimeric flavivirus formulations can be delivered by different routes of administration, e.g., systemically, regionally, or locally. Regional administration refers to administration into a specific anatomical space, such as intraperitoneal, intrathecal, subdural, or to a specific organ, and the like. Local administration refers to administration of a composition into a limited, or circumscribed, anatomic space such as subcutaneous injections, intramuscular injections, intradermal injections, or by application to the epidermis. One of skill appreciates that local administration or regional administration can also result in entry of the viral preparation into the circulatory system. Typical delivery routes include parenteral administration, e.g., intradermal, intramuscular or subcutaneous routes. Other routes include oral administration, including administration to the oral mucosa (e.g., tonsils), intranasal, sublingual. For delivery of a chimeric flavivirus, administration can also be performed via inhalation. Aerosol formulations can, for example, be placed into pressurized, pharmaceutically acceptable propellants, such as dichlorodifluoro-methane, nitrogen and the like. They can also be formulated as pharmaceuticals for non-pressurized preparations such as in a nebulizer or an atomizer. Typically, such administration is in an aqueous pharmacologically acceptable buffer as described above.

The vaccines of the invention can be administered in a variety of unit dosage forms, depending upon the intended use, e.g., prophylactic vaccine or therapeutic regimen, and the route of administration. With regard to therapeutic use, the particular condition or disease and the general medical condition of each patient will influence the dosing regimen. The concentration of chimeric flavivirus in the pharmaceutically acceptable excipient can be, e.g., from about 5 μg to about 100 μg of chimeric flavivirus per dose, between about 15 μg to about 85 μg of chimeric flavivirus per dose, between about 25 μg to about 75 μg of chimeric flavivirus per dose, between about 35 μg to about 65 μg of chimeric flavivirus per dose, or between about 45 μg to about 55 μg of chimeric flavivirus per dose.

The amount and concentration of virus and the formulation of a given dose, or a “therapeutically effective” dose can be determined by the clinician. A therapeutically effective dose of a vaccine is an amount of chimeric flavivirus that will stimulate an immune response to the structural protein(s) encoded by the dengue virus or yellow fever virus nucleic acid included in the chimeric flavivirus vector. The dosage schedule, i.e., the dosing regimen, will depend upon a variety of factors, e.g., the general state of the patient's health, physical status, age and the like. The state of the art allows the clinician to determine the dosage regimen for each individual patient.

Single or multiple administrations of the chimeric flavivirus vectors and/or chimeric flavivirus formulations can be administered as prophylactic vaccines. In one embodiment, multiple doses (e.g., two or more, three or more, four or more, or five or more doses) are administered to a subject to induce or boost a protective immune response. The two or more doses can be separated by periodic intervals, for instance, one week, two week, three week, one month, two month, three month, or six month intervals. In an exemplary embodiment, only two doses of the vaccine are required and may be administered at short intervals (e.g., two weeks apart, three weeks apart, or four weeks apart).

In yet another aspect, the invention also provides kits that contain the chimeric flavivirus vectors, chimeric flaviviruses, or vaccines of the invention. The kits can, for example, also contain cells for growing the chimeric flaviviruses of the invention. The kits can also include instructional material teaching methodologies for generating chimeric flaviviruses using the kits and, for vaccines, can include instruction for indication of dosages, routes and methods of administration and the like. In one embodiment, kits include containers suitable for transport and/or store flavivirus vectors, chimeric flaviviruses, or vaccines of the invention.

The following examples illustrate various aspects of the present invention. The examples should, of course, be understood to be merely illustrative of only certain embodiments of the invention and not to constitute limitations upon the scope of the invention which is defined by the claims that are appended at the end of this description.

### EXAMPLES

### Example 1

### Vector Selection

A search was conducted of the published literature and data in the Catalogue of Arthropod-Borne Viruses (CDC, Division of Vector-Borne Infectious Diseases, Arbovirus Catalog) to find candidate BSL2 viruses with the potential to replicate to high titer in Vero (African green monkey kidney) cells, a continuous cell line commonly used for production of licensed vaccines, e.g., against Japanese encephalitis, smallpox, rabies, and polio. The search identified very few virus candidates. Three BSL2 viruses were selected by virtue of high growth potential belonging to a cluster of three phylogenetically related mosquito-borne viruses in the Edge Hill subgroup [Uganda S (UGS), Banzi (BAN), and Jugra (JUG)] (Grard et al., 2010, J Gen Virol 91: 87-94). A fourth BSL2 virus [Rio Bravo virus (RBV)] belongs to a distinct and different grouping of Flaviviruses having no known arthropod vector. West Nile (WN) virus (BSL3), which is known to grow to high titer (9 log₁₀ PFU/mL) was selected as a comparator.

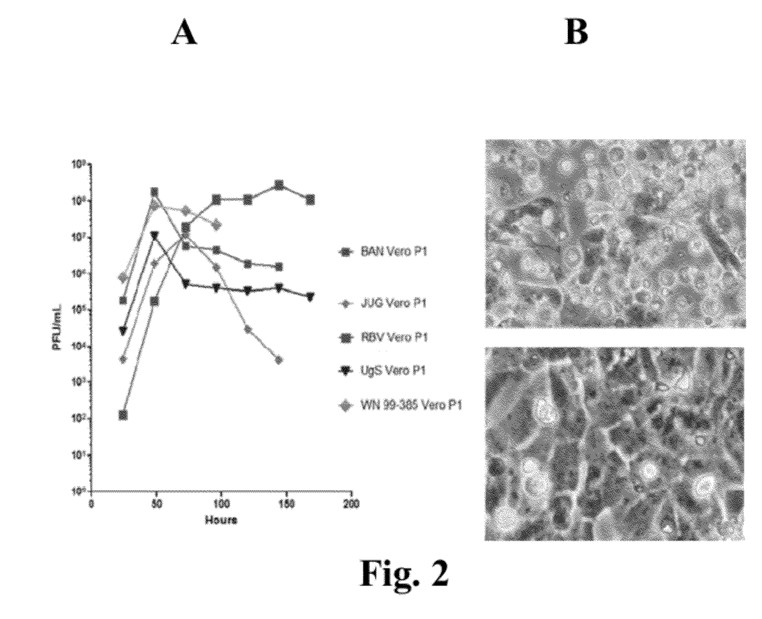

Seed stocks of the lowest passage of the candidate viruses available in the World Reference Center for Arboviruses (University of Texas Medical Branch, Galveston Tex.) were prepared in Vero (WHO 10-87) cells. To compare the growth of these flaviviruses, Vero cells (WHO 10-87, passage 141) and A549 cells (passage 21) were first grown in monolayer cultures in 25 cm² flasks using OptiPro® serum free medium (Gibco), and infected with virus at MOI of 0.01 plaque-forming units (PFU)/cell. Virus yields in A549 cells were significantly lower than in Vero cells (data not shown). The kinetics of virus replication of various potential vectors in Vero cells is shown in FIG. 2. RBV achieved high yields (>8 log 10 PFU/mL) without causing cytopathic effects (CPE). In contrast to the results seen with RBV, typical of most flaviviruses, WN, UGS, BAN and JUG viruses caused extensive CPE beginning within a few hours of peak virus yields, and as the cells ceased macromolecular synthesis and underwent lysis or apoptosis, virus titers in the supernatant medium fell off rapidly (FIG. 2A). During replication, flaviviruses are released as mature, infectious virions into the cell culture medium; since they are unstable and lose infectivity rapidly outside of the cell at temperatures used for growth (37° C.), the virus must be harvested at the peak titer, a critical, single point in time (within a period of 6-8 hr) which occurs when early CPE is evident. In contrast, RBV did little damage to cells, while still producing a high titer of virus (FIG. 2B). Moreover, replication of RBV continued for up to 7 days with continued release of high titers of virus.

Persistent infection of cell cultures with RBV has been described previously, and is not due to higher thermostability of virus in cell culture medium (Wilhelm et al., 1970, Appl Microbiol 20: 612-5). This property of RBV may be related to the natural ecology of the virus. Rio Bravo virus was originally isolated from Mexican free-tailed bats in 1954. The virus has not been extensively studied, but it is known to cause persistent infections of salivary glands of bats (Burns et al., 1956, Science 123: 227-8; Considine et al., 1964, Pub Hlth Rep 79: 1033-9). The unusual trait of high-titer persistent infection in vitro can be harnessed for optimized vaccine manufacturing, since multiple harvests of the cell culture supernatant (as opposed to one carefully timed harvest) can be performed. Moreover, due to the absence of CPE, host cell protein (HCP) and DNA release into the cell culture medium is minimized, facilitating removal of these residual contaminants from the drug substance. Adaptation by serial passage resulted in higher yields (≧9 log 10 PFU/mL) of RBV, as described below.

The specific Rio Bravo virus used as the vector for constructing a chimeric virus may be one of the extant strains, including the Burns Bat strain (Texas, 1954), M64 (derived from the Burns bat strain), TVRL 126865) (Trinidad, 1973) or RiMAR (GenBank Accession AF 144692). As an example, the specific strain used to determine the potential of Rio Bravo virus as a vector was the M64 strain. M64 is considered the “prototype” strain of Rio Bravo virus (Catalogue of Arthropod-Borne Viruses, op cit).

The nucleotide and amino acid sequence of the M64 strain was determined and compared with the only published sequence of Rio Bravo virus (the RiMAR strain). Genomic viral RNA (vRNA) was isolated with the Qiagen viral RNA isolation kit. The 5′ and 3′-terminus sequences of the specific genomes were determined by their decapping, ligation and sequencing and using the Ambion FirstChoice RLM RACE kit. Initial overlapping cDNA fragments and amplicons of the Rio Bravo virus, M64 strain, were generated using primer pairs and primers specific to the published sequence of Rio Bravo virus, RiMar strain (Charlier et al., 2002, J Gen Virol 83: 1875-85; Genbank Accession number AF144692) by utilizing the one-step reverse transcriptase polymerase chain reaction (RT-PCR) (Roche Diagnostics), in four overlapping cDNA fragments containing genome nucleotide regions 100-3514, 2555-4955, 4551-7609, 7009-10020. Prior hereto, only a partial (NS5) sequence of the M64 strain had been sequenced (Billoir et al., 2000, J Gen Virol 81: 781-90). First strand synthesis was performed at 50° C. for 30 minutes, whereas amplification underwent a total of 35 cycles with annealing temperature set at 5° C. below the lowest melting temperature (Tm) of the PCR primer pairs and extension set at one minute per 1000 nucleotides. Amplified sequences were gel purified and automated sequencing with specific sequencing primers for both strands provided consensus sequences. 21 nucleotide differences located throughout the open reading frame (ORF) between M64 passage 1 and RiMAR strains were observed, and only one nucleotide difference occurred in the 3′ NCR.

TABLE 1
Nucleotide and amino acid differences between
RiMAR (Genbank Accession number AF144692)
and M64 strains of Rio Bravo virus.

| | | Nucleotide | Amino Acid |
|---|---|---|---|
| Gene | Site¹ | substitution² | substitution³ |
| C | 268 | C → A | T → K |
| E | 1113 | A → T | T → S |
| E | 1407 | C → T | L → F |
| E | 1489 | C → T | A → V |
| E | 1727 | T → C | — |
| NS2A | 3534 | C → T | P → S |
| NS3 | 5760 | T → C | — |
| NS3 | 6170 | T → G | I → M |
| NS4A | 6500 | T → C | — |
| NS5 | 7677 | C → T | P → S |
| NS5 | 7802 | C → T | — |
| NS5 | 7805 | G → A | — |
| NS5 | 7808 | C → A | — |
| NS5 | 7811 | C → T | — |
| NS5 | 7814 | C → A | — |
| NS5 | 7815 | C → A | — |
| NS5 | 7817 | C → G | — |
| NS5 | 7820 | C → A | — |
| NS5 | 7823 | C → T | — |
| NS5 | 8931 | A → T | M → L |
| NS5 | 9557 | A → G | — |
| 3′- NCR | 10299 | G → A | — |

¹Indicates nucleotide location from the start of the virus genome
²Arrow represent directionality of the mutation. On the left represents nucleotide present on RiMAR strain and on the right nucleotide present on the M64 strain
³Arrow represent directionality of the mutation. On the left represents amino acid present on RiMAR strain and on the right amino acid present on the M64 strain

To determine whether the absence of lytic infection of cells infected with Rio Bravo virus was associated with release of host cell DNA into the medium, samples of medium were taken daily from monolayer cultures (25 cm² flasks) of Vero cells infected with Rio Bravo (‘adapted’, P10) and Banzi viruses. Banzi virus produces lytic cytopathic effects, whereas Rio Bravo does not (FIG. 2). DNA in the culture medium was measured by picogreen assay (Quant-iT PicoGreen dsDNA kit, Invitrogen). The results are shown in Table 2; Rio Bravo infection did not cause an elevation in host cell DNA (compared to mock infection), whereas Banzi virus resulted in a substantial extracellular DNA release on Day 3 after infection, corresponding to the appearance of lytic CPE. The results indicate that Rio Bravo virus (or its chimera) could be harvested for 5 days or more, corresponding to peak levels of virus in the medium (FIG. 2A) with minimal contamination with host cell DNA.

TABLE 2
Vero Cell DNA in Medium from Cultures Infected
with Rio Bravo and Banzi Viruses.

| | DNA (ng/mL) | | | |
|---|---|---|---|---|
| Virus | Day 1 | Day 2 | Day 3 | Day 5 |
| Mock | 425 | 428 | 447 | 420 |
| Rio Bravo P10 | | 396 | 406 | 412 |
| Banzi P1 | 653 | 494 | 6439 | Not tested (CPE) |

### Example 2

### Adaptation to the Cell Substrate

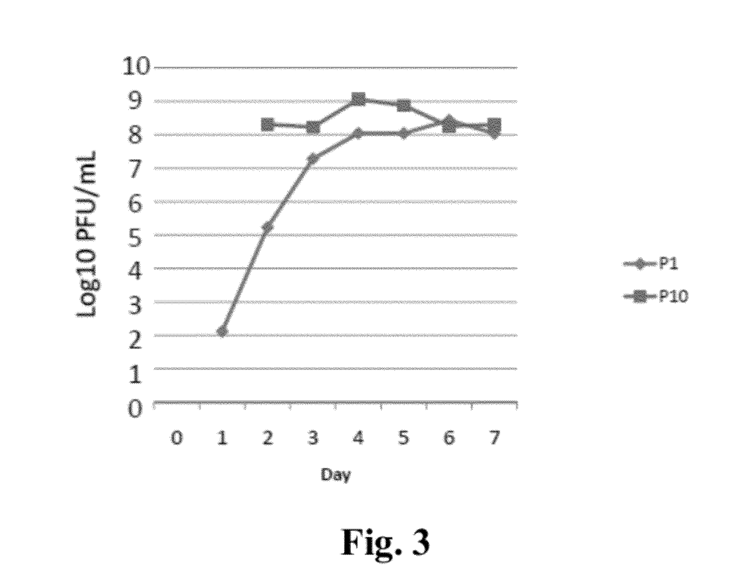

This example illustrates how a Rio Bravo virus strain is adapted to increase the yield (titer) of virus in cell cultures. The stock culture of Rio Bravo virus was passed by infecting duplicate monolayer culture of Vero cells in 25 cm² flasks at MOI 0.01. Blind passages were subsequently made of virus harvested in the early phase of growth cycle. At each passage, cell culture medium was removed from the flask, 0.1 mL of the virus to be passed is added to the flask and allowed to adsorb for 1 hr at 37° C., and the culture is washed to remove the inoculum, after which fresh medium is added. At each passage, 3 aliquots of each virus were frozen for titration. When the growth kinetics of adapted [10th passage (P10)] of Rio Bravo virus was compared to the growth curve of original virus stock, it was observed that the peak titer had increased approximately 5-fold to >10⁹ PFU/mL (FIG. 3). Cytopathic effects were absent or minimal with no differences between the two passage levels; i.e., in both cases persistent infection with prolonged virus yields was observed.

The full genomic sequence of the 1^{st} and 10^{th} passage Rio Bravo virus were then compared to determine whether there were mutations associated with the increase in virus titer in Vero cells observed in the ‘adapted’ P10 virus. The same strategy described above for sequencing the RBV M64 genome was employed to determine the complete ORF sequence of the P10-Vero 10-87 adapted M64 strain. Comparison of P1 and P10 M64 strain indicated 2 nucleotide changes, of which C1489T is located within the Envelope (E) gene (Table 3). This mutation is within Domain II, a finger-like structure comprised of a pair of discontinuous loops one of which is highly conserved among all flaviviruses functioning as an internal fusion peptide and stabilized by three disulfide bridges.

TABLE 3
Nucleotide and amino acid differences between
M64 Passage 1 (P1) and Passage 10 large plaque
phenotype (P10) strains of Rio Bravo virus.

| | | Nucleotide | Amino Acid |
|---|---|---|---|
| Gene | Site¹ | substitution² | substitution³ |
| E | 1489 | C → T | A → V |
| NS3 | 6170 | T → G | I → M |

¹Indicates nucleotide location from the start of the virus genome
²Arrow represent directionality of the mutation. On the left represents nucleotide present on P1 and on the right nucleotide present on P10 the M64 strain
³Arrow represent directionality of the mutation. On the left represents amino acid present on P1 strain and on the right amino acid present on P10 the M64 strain

It was also observed that the Rio Bravo virus contained two distinct plaque populations, a small plaque virus (1 mm) and a large plaque population (5-6 mm). The two plaque populations were seen at all passage levels (P1 through P10). The two plaque populations were separated by picking plaques in uncrowded cultures (two rounds) followed by a round of terminal dilution cloning. The small and large plaque populations were then amplified by passage in monolayer cultures of Vero cells in serum free medium. The small plaque population, as a pure culture, has a slightly larger plaque size (2-3 mm) compared to the original small plaque (1 mm), whereas the large plaque population did not change (5-6 mm) Full genomic sequencing revealed one 3-nucleotide deletion mapped within Domain II of the E protein, three, additional mutations resulting in amino acid changes in the E gene, two amino acid mutations in non-structural genes (NS1 and NS3) and one nucleotide deletion in the 3′-NCR (Table 4).

TABLE 4
Nucleotide and amino acid differences between M64
Passage 1 (P1) and Passage 10 small plaque phenotype
(P10 S.P.) strains of Rio Bravo virus.

| | | Nucleotide | Amino Acid |
|---|---|---|---|
| Gene | Site¹ | substitution² | substitution³ |
| E | 1485-88 | AGT (deletion) | S → Δ |
| E | 1518 | C → T | L → F |
| E | 1660 | A → C | E → A |
| E | 1663 | G → A | C → Y |
| NS1 | 3312 | C → T | P → S |
| NS3 | 6131 | A → G | — |
| NS3 | 6169 | T → C | I → T |
| 3′-NCR | 10692 | T (deletion) | — |

¹Indicates nucleotide location from the start of the virus genome
²Arrow represent directionality of the mutation. On the left represents nucleotide present on P1 and on the right nucleotide present on P10 the M64 strain
³Arrow represent directionality of the mutation. On the left represents amino acid present on P1 strain and on the right amino acid present on P10 the M64 strain

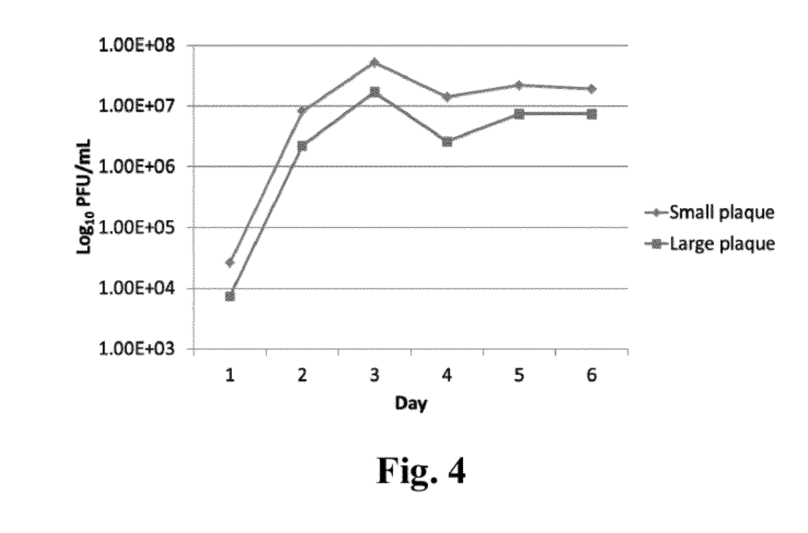

To determine if there was a difference in growth kinetics between small and large plaque populations, growth curves at MOI 0.01 were performed in Vero cells. The small plaque population showed 0.5 log₁₀ (3-fold) higher growth in Vero cells than the large plaque (FIG. 4). The difference could have been due to one or more of the mutations in either structural, non-structural genes and or 3′-NCR. Although, the precise function of the nonstructural protein 1 (NS1) it not well defined, it is possible that the non-synonymous mutation at nucleotide 3312 may contribute to the increased growth of the small plaque phenotype in Vero cells. Additionally, either of the 2 mutations present in the nonstructural gene 3 (NS3) (Table 4), may affect the functions of any of the enzyme activities such as trypsin-like serine protease, helicase, and RNA triphosphatase (RTPase) that are involved in the processing of the polyprotein and RNA replication. Lastly, the deletion at the 3′-NCR is located within the highly conserved stem and loop structure (3′-SL) which plays a significant role during viral replication by facilitating the binding of cellular and/or viral factors to the 3′-SL.

### Example 3

### Selection of the Cell Substrate

To select the optimum cell culture for growth of Rio Bravo virus (or chimeric viruses derived therefrom), the virus was used to infect different mammalian cell lines acceptable for manufacturing human vaccines. These included Vero (WHO 10-87), fetal rhesus lung (FRhL), A549, and Madin Darby Canine Kidney (MDCK) cells, all infected at MOI 0.01 PFU/cell. Vero cells produced the highest peak yield of virus as shown in Table 5.

TABLE 5
Yields of Rio Bravo Virus in Different Cell Types.

| Cell type | Peak titer (PFU/mL) |
|---|---|
| Vero (WHO 10-87) | 1.2 × 10⁹ |
| A549 | 1.7 × 10⁶ |
| FRhL | 1.1 × 10⁶ |
| MDCK | 2.6 × 10³ |

### Example 4

### Selection of the Envelope Gene Donor Strain

To select the optimum strain of dengue virus to be used as a pre-membrane and envelope gene donor in construction of a chimeric virus, multiple strains of dengue virus were compared for growth in the selected substrate for manufacture (Vero cells). The hypothesis proposed is that by selecting a Flavivirus vector with highest growth, a cell line with highest permissiveness, and a donor (dengue) virus with highest growth capacity the resulting chimeric virus would also grow to high titers.

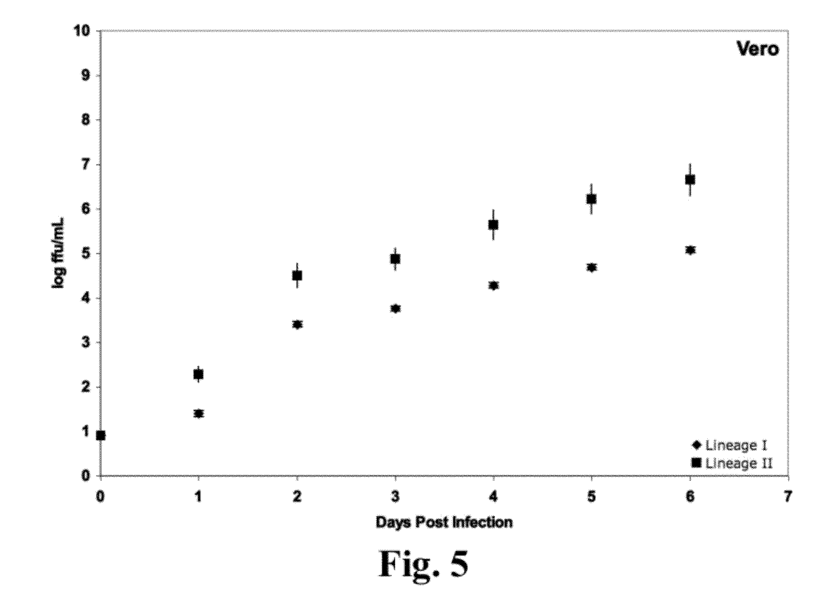

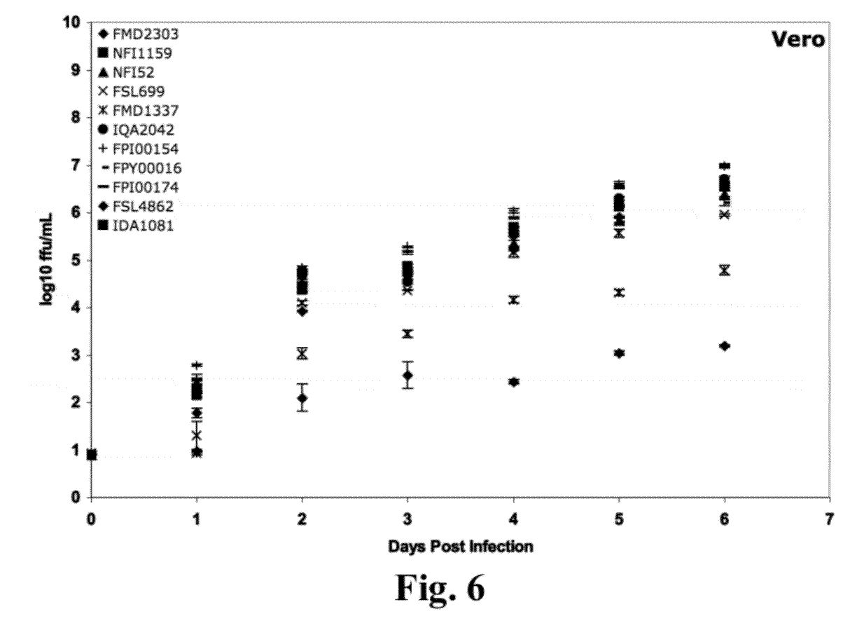

To search for dengue virus with highest replication in Vero cells as a gene donor for construction of chimeric virus, virus strains at the lowest possible passage level were obtained from various sites engaged in field assessments of dengue epidemiology. Strains were isolated from blood of patients with dengue disease. All dengue serotypes are differentiated by sequencing into different lineages or clades (Weaver et al., 2009, Infection, Genetics and Evolution 9: 523-540; Holmes et al., 2003, Infection, Genetics and Evolution 3: 19-28). Initially, dengue type 2 was investigated as a gene donor. A collection of recent isolates virus from human dengue cases in Peru were passed once in C6/36 mosquito cells and used to measure replication kinetics in Vero 10-87 cells. A new lineage of dengue-2 (American/Asian genotype) associated with severe disease and likely to represent a more virulent strain was recently described in Peru (Mamani, et al., 2011, Rev Peru Med Exp Salud Pub 28: 72-77), and many of the strains investigated were of this lineage. Growth in Vero cells was investigated, and the analysis showed that the new lineage II strains replicated to higher titer than the older lineage I strains circulating in this region (FIG. 5). The virus employed in the vaccine construction could be any of the high-yielding strains resulting from a detailed analysis of individual strains in Lineage II (FIG. 6). Some strains (in Lineage II) grew to relatively high titer (>10⁶ PFU/mL). Strain FPI00154 was selected as a donor strain for construction of chimeric virus.

### Example 5

### Construction of Vector Infectious Clone

The description below shows how the Rio Bravo virus strain is cloned for use in constructing a chimera. These principles can also be applied to any other flavivirus described herein, including, but not limited to, the Uganda S virus, Banzi virus, Jugra virus, or any mosquito-associated Flavivirus. In an example, a cDNA copy of the entire 11-kilobase genome of the selected Rio Bravo Virus P10 is used to construct the chimera. Since the full length sequence may be unstable, the RBV genome is expressed under the influence of a cytomegalovirus (CMV) promoter and the incorporation of a stabilizing intron sequence is engineered at the junction of envelope (E) and the non-structural 1 (NS1) gene. The Rio Bravo virus-specific pre-membrane (prM) and envelope (E) genes within the full length infectious clone (FLIC) are replaced by the prM-E sequences of a selected dengue virus serotype using standard molecular cloning (including fusion PCR) techniques. Rescue of the chimeric RB-DEN virus is obtained by transfection by electroporation or lipofectamine into Vero 10-87 cells and progeny virus is harvested by collection of the supernatant. Suitable passages are made to produce master and working virus seeds.

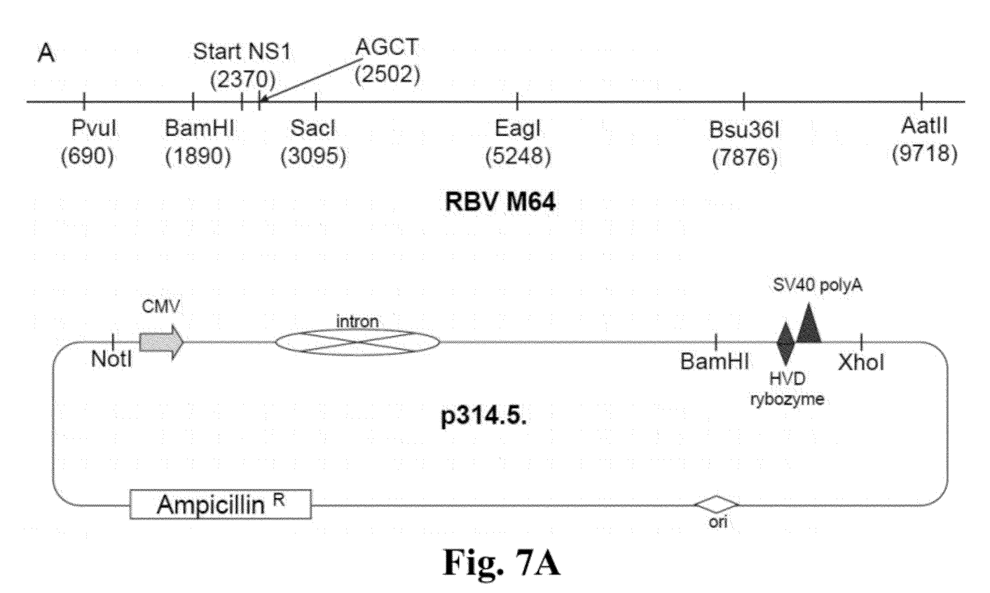

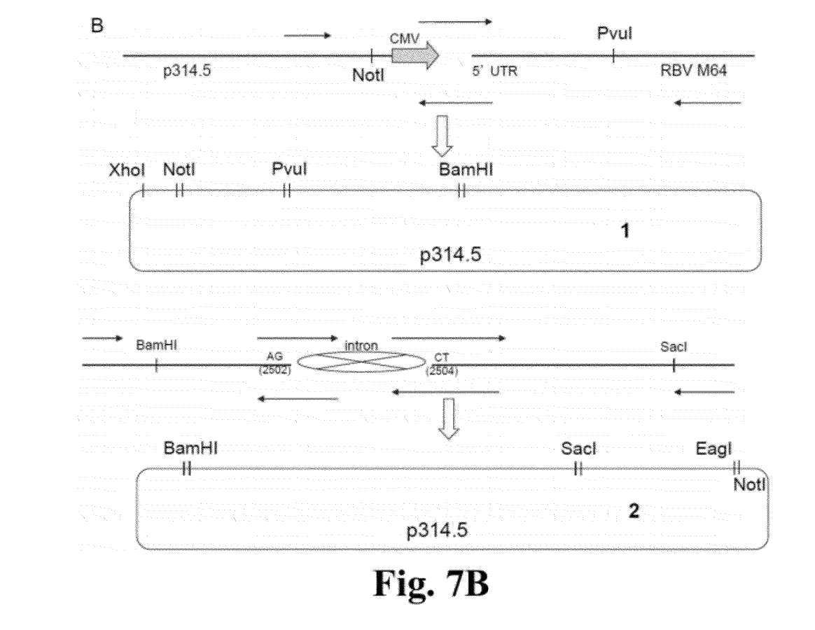

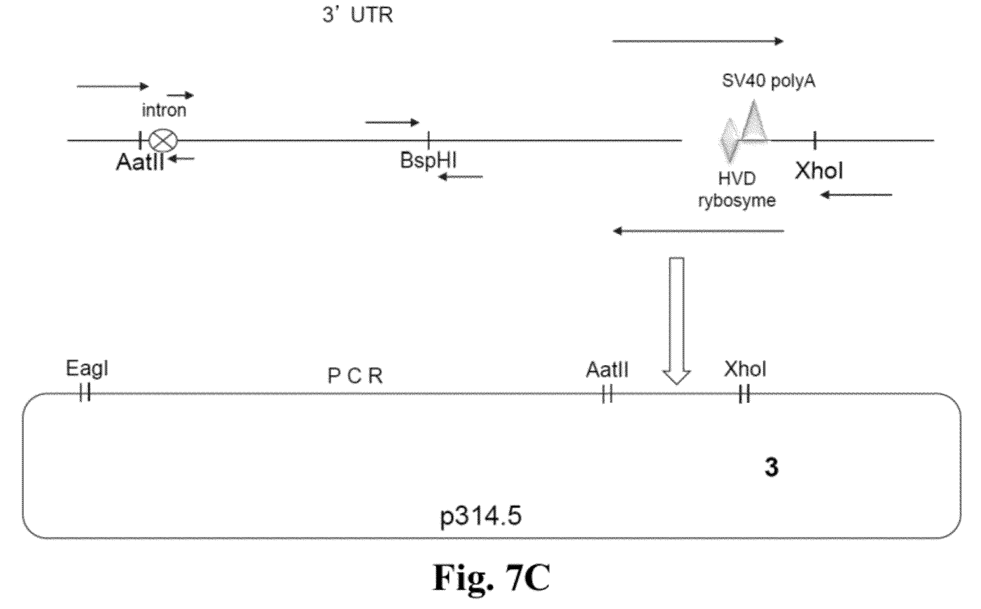

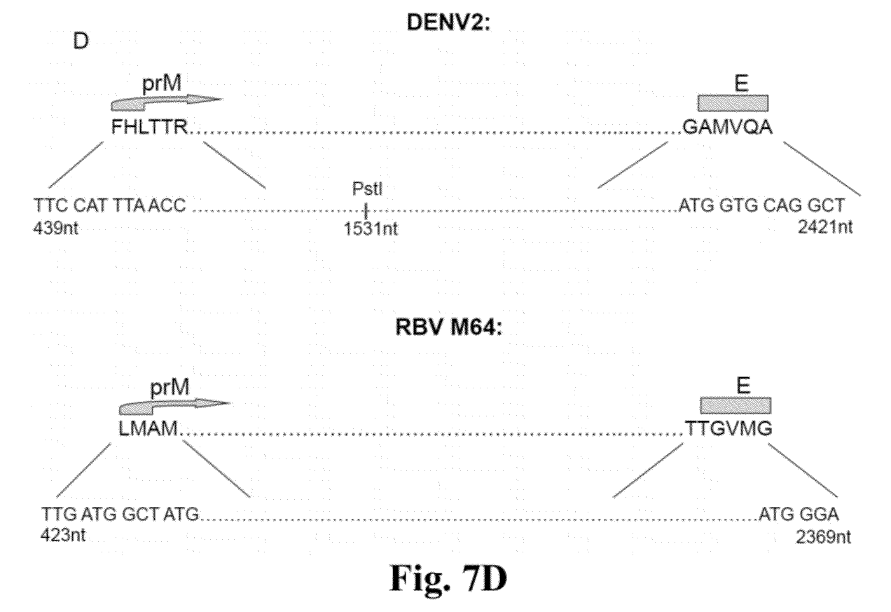

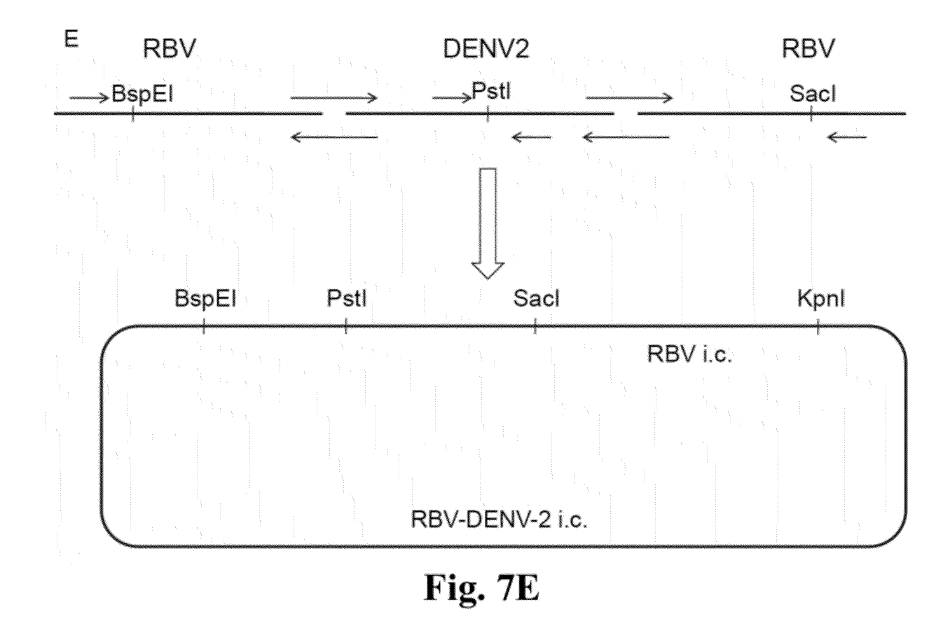

Initially a full-length infectious clone of RBV is constructed. The complete RBV genomic sequence is propagated in one plasmid under the expression of the cytomegalovirus (CMV) promoter at the 5′ end. A unique feature of the infectious clone is the insertion of the hepatitis δ virus ribozyme (HDVr) immediately after the last nucleotide of Rio Bravo Virus cDNA sequence to ensure production of Rio Bravo Virus RNAs with the precise 3′-terminus, which is beneficial for more efficient RNA replication. Furthermore, a SV40 polyadenylation site can be inserted downstream of the HDVr to ensure complete termination of transcription (FIG. 7A). Five cDNA fragments containing the regions 1-1890 (fragment 1, NotI/BamHI), 1890-5248 (fragment 2, BamHI/EagI) and 5248-7876 (fragment 3, EagI/Bsu361), 7876-9718 (fragment 4, Bsu36I/AatII) and 9718-10742 (fragment 5, AatII/XhoI) overlapping by a minimum of 100 nucleotides and spanning the entire genome are amplified with strain specific primer pairs, ligated individually into plasmids 1-5 and sequentially into the polylinker of the pACNR-based low-copy plasmid (FIGS. 7B and 7C). Plasmid 2 contains a 300 nucleotide-long intron inserted by fusion PCR at the junction of the envelope (E) and non-structural gene 1 (NS1) to ensure stability of the resultant full-length cDNA template (FIG. 7B). Plasmid 5 contains the hepatitis δ virus ribozyme (HDVr) immediately after the last nucleotide of Rio Bravo Virus cDNA sequence, a SV40 polyadenylation site downstream of the HDVr, as well as a second intron inserted at nucleotide 9742 to increase stability of the construct, which is generated by fusion PCR (FIG. 7C). The full-length cDNA template is generated sequentially by ligation with appropriate restriction fragments derived from these plasmids. To generate the chimeric RB-DEN cDNA sequences, the RBV prME sequences are replaced by the corresponding DEN2 prME sequences that are produced by RT-PCR amplification of the specific virus genome, followed by fusion PCR to generate an amplicon incorporating the complete DEN2 prME sequence as well as RBV sequences spanning the BspEI and Sad sites (FIG. 7D). The chimeric RBV-DEN2 cDNA sequence is generated by digestion of the RBV cDNA and RB-DEN2 amplicon with the BspEI and Sad restriction enzymes and ligation into the plasmid containing the RBV BspEI/SacI digested cDNA (FIG. 7E). The resulting virus is sequenced to confirm the expected genome. The signal sequence for furin proteolysis which represents the junction at the 5′ end of the DEN2 donor prM gene is critical to successful generation of a viable chimera. It is thus ensured that the RBV vector signal sequence is retained in the recombinant infectious clone. The recombinant infectious clone may be transfected by lipofection or electroporation into Vero 10-87 cells. Positive-sense RNA is then produced from the CMV promoter embedded upstream of the virus gene coding region in a run-off transcription reaction. Since Flaviviruses are positive-sense and their genomic RNA can serve directly as message for translation of proteins required for virus replication, this reaction produces chimeric DEN2/RBV RNA transcripts that are infectious and are capable of producing progeny virions in host cells. During replication the larger pr segment is released into the extracellular medium, although prM/M cleavage is sometimes incomplete so that some prM remains in virions. Antibodies to prM may be undesirable in the case of DEN, since they can play a role in immune enhancement. For that reason, construction of a chimeric virus containing only DEN E protein can also be performed. The E-only construct is used if growth is not inferior to the alternative prM-E chimera. The structure of the assembled Flavivirus virion and many Flavivirus proteins have been determined by x-ray crystallography and/or nuclear magnetic resonance; each virus particle contains a multi-copy presentation of 180 copies of the E protein with identical amino acid sequence assembled in a dimeric head to tail configuration. The E protein copies contain both type-specific and cross-reactive DEN neutralizing epitopes required to elicit protective immunity. The NS proteins, in this case encoded by the RBV vector remain either in the cell or (if soluble and released, like NS1), are removed during vaccine purification. Once the chimeric genome RNA is available, cells are transfected for preparation of a Research Master Seed (P2 after transfection).

Chimerization is known to attenuate virulence of Flaviviruses and may or may not affect replication in vitro. The experience with DEN/yellow fever chimeric vaccine, for example, showed a reduction in neurovirulence of the chimera compared to the parental YF 17D backbone, but did not impair replication in Vero cells. To optimize replication of the DEN 2/RBV chimeric virus in the cell substrate selected for manufacturing, the virus is adapted by 10 sequential passages. This passage series also assesses genetic stability of the chimera. Growth kinetics are used to compare the unpassaged and P10 virus; it is expected that approximately 0.5 log₁₀ higher yields will result (e.g., See FIG. 3). Sequencing will be performed to determine the mutation(s) associated with enhanced growth and these are introduced into the infectious clone by site-directed mutagenesis. A new Research Virus Seed (P10+) is manufactured. Growth kinetics at MOI 0.1 to 0.001 are evaluated and the optimal MOI selected. The seed virus is used to infect cells grown in stationary cultures (T 225 flasks), and culture fluid harvested and partially purified by removing cell debris (0.8μ filter), concentrating the virus by ultrafiltration/diafiltration (100 kDa filter), and inactivating the virus with 0.1%-propiolactone or another suitable method of inactivation. Absence of residual live virus will be determined by inoculating Vero indicator cells (2 blind passages, 7 days apart) and performing plaque assays (by immunohistochemistry) on cell culture supernate. Vaccine antigen potency is determined by dengue-specific monoclonal ELISA standardized to the plaque assay for live virus. Vaccine is frozen and formulated with adjuvants immediately prior to administration to mice.

Donor DEN 1, 3 and 4 strains (human isolates, low passage) are selected based on high growth in Vero (or another cell substrate selected as described). Similar cloning templates to the ones used to generate a high-yield DEN2 (prM-E)/RBV chimera are used to construct chimeras containing DEN 1, 3 and 4 prM-E (or E gene if viable for DEN2/RBV), with strict observance of gene junction sequences, particularly the prM signal sequence. RBV genomic sequences are propagated in one plasmid (FIG. 7A). As is done for the DEN 2 chimera, RBV sequences within the RBV infectious clone are replaced by the corresponding DEN1, 3 and 4 prME or E sequences that are produced by RT-PCR amplification and fusion of the specific virus genome, resulting in the generation of RBV DEN 1 (3 or 4) prM-E and RBV DEN 1 (3 or 4) E plasmids (FIGS. 7D and 7E). These plasmids may be modified at specific positions by site-directed mutagenesis to introduce specific mutations associated with adaptation to Vero (or another cell substrate). The corresponding RBV-DEN1, 3 and 4 plasmids are used to transfect the cell substrate of choice by electroporation or lipofectamine. The resulting virus is sequenced to confirm the expected genome.

The viability of each construct and growth kinetics in the selected cells for manufacture is defined, and if necessary, adaptation for high growth may be performed as described above for the P1→P10 adaptation of RBV. This work generally proceeds in a predictable manner, based on experience with the DEN 2/RBV construct. Growth is optimized to ensure that the viruses generated can be used for manufacturing by (i) adaptation (serial passage) and/or (ii) inserting mutations (by site directed mutagenesis of the infectious clone) in the RBV vector backbone known to be associated with higher growth. These mutations may include those shown in Tables 3 and 4 in the nonstructural or 3′-NCR of Rio Bravo virus. Research Seed Viruses are made and potency (PFU/mL) and identity confirmed by sequencing.

### Example 6

### Preparation of Inactivated Vaccine

The methods for production of inactivated vaccine are those well known in the art, and have been described in patents and patent applications, and various publications (e.g., WO/2006/122964; Srivastava et al., 2001, Vaccine 19: 4557-65). Briefly, cells grown in suspension, on microcarrier beads, or on the surface of roller bottles or cell factories are infected with working seed virus at the appropriate MOI. Virus is harvested from cell cultures at the appropriate time after infection determined by growth curve studies, generally when CPE is just beginning. For example, the virus harvest is clarified by passage through a depth filter, digested with Benzonase® (nuclease) and ultrafiltered/diafiltered to remove host DNA. Given the low levels of host cell DNA in cell culture medium following infection of RBV (or chimeric den/RBV) virus, it may be acceptable to eliminate Benzonase® digestion. Additional purification steps can employ methods well known in the art including protamine sulfate precipitation, cellufine sulfate chromatography, anion-exchange and size exclusion chromatography, and sucrose gradient centrifugation. Inactivation of the virus is achieved either before, between steps, or after purification.

Methods that can be used to inactivate the dengue/Rio Bravo or dengue/Uganda S subgroup chimeric viruses include treatment with chemicals (such as, but not limited to formalin, beta-propiolactone, aziridines (e.g. ethyleneimine), hydrogen peroxide, organic solvents, and ascorbic acid), high pressure, ultraviolet radiation with or without psoralen sensitization or gamma irradiation. These methods invariably degrade the viral RNA by alkylation or other reactions. In contrast, the dengue-mosquito Flavivirus chimera is incapable of replication in mammalian cells and can be used without chemical or other means of inactivation, preserving the structure of the viral RNA.

Application of these methods yields a purified, inactivated dengue vaccine with a potency of greater than or equal to 10⁹ PFU in the form of inactivated virions per 0.5 mL of vaccine, 0.5 mL representing the volume typically injected into humans. Preferably the method will yield a potency of 10¹⁰ PFU in the form of inactivated virions per 0.5 mL. The equivalent amount of viral envelope protein in the formulation expressed in micrograms is greater or equal to 10 micrograms/0.5 mL dose. Preferably the method will yield a potency of 20 micrograms of viral protein, or greater/0.5 mL. A potency in the range of 10⁹ whole virions/mL or 10 micrograms is not reproducibly achievable by standard methods of propagating dengue virus itself and potencies of 10¹⁰ whole virions or 20 micrograms per human dose cannot be achieved by standard methods.

Similar methods of producing chimeric inactivated vaccines can be applied to the manufacture of high-titer inactivated vaccines with a potency of ≧10⁹ PFU (equivalent as inactivated virus) or ≧10 micrograms of protein per 0.5 mL against other Flaviviruses that are difficult to propagate to high titer in cell cultures, such as yellow fever 17D virus.

To the extent that any definitions in documents incorporated by reference are inconsistent with the definitions provided herein, the definitions provided herein are controlling. Although the invention has been described with reference to the presently preferred embodiments, it should be understood that various changes and modifications, as would be obvious to one skilled in the art, can be made without departing from the spirit of the invention. Accordingly, the invention is limited only by the following claims.

The disclosures, including the claims, figures and/or drawings, of each and every patent, patent application, and publication cited herein are hereby incorporated herein by reference in their entireties.

What is claimed:

1. A chimeric flavivirus vector encoding
a structural protein from a first flavivirus with a low level of replication in a cell and
a backbone from a second flavivirus with a high level of replication in the cell,
wherein the structural protein is an envelope protein and the backbone comprises the capsid protein, the non-structural proteins and the 3′ and 5′ noncoding termini, and
wherein the second flavivirus is a flavivirus selected from the group consisting of the Rio Bravo taxonomic group, the Uganda S taxonomic group, and the mosquito-associated flaviviruses.

2. The chimeric flavivirus vector of claim 1, wherein the structural protein comprises an envelope protein and a pre-membrane protein of the first flavivirus.

3. The chimeric flavivirus vector of claim 1, wherein the first flavivirus is a dengue virus or a yellow fever virus.

4. The chimeric flavivirus vector of claim 1, wherein the first flavivirus is a dengue virus selected from the group consisting of dengue type 1 (DEN1) virus, dengue type 2 (DEN2) virus, dengue type 3 (DEN3) virus, and dengue type 4 (DEN 4) virus.

5. The chimeric flavivirus vector of claim 1, wherein said Rio Bravo taxonomic group is selected from the group consisting of the Rio Bravo (sensu stricto) virus, the Montana Myotis Leukoencephalitis virus, the Dakar Bat virus, the Phnom Penh Bat virus, the Carey Island virus, and the Bukalasa Bat virus.

6. The chimeric flavivirus vector of claim 1, wherein said Uganda S taxonomic group is selected from the group consisting of the Uganda S virus, the Banzi virus, and the Jugra virus.

7. The chimeric flavivirus vector of claim 1, wherein said mosquito-associated flavivirus is selected from the group consisting of the Kamiti River virus, the Culex flavivirus, the Aedes flavivirus, the Nakiwogo virus, the Quang Binh virus, and the Cell Fusing Agent virus.

8. The chimeric flavivirus vector of claim 1, wherein the backbone is selected from a flavivirus adapted to a cell substrate.

9. The chimeric flavivirus vector of claim 8, wherein the backbone is selected from a flavivirus adapted to a cell substrate such that an increase in yield of the flavivirus is at least about 2.5-fold as compared to the yield of the flavivirus before adaptation.

10. The chimeric flavivirus vector of claim 1, wherein the backbone is selected from a flavivirus adapted to Vero, Madin Darby Canine kidney (MDCK), A549, fetal rhesus lung (FRhL), MRC5, human embryonic kidney (HEK293), primary dog kidney, primary rabbit kidney, xenopus oocyte, or chick embryo cells.

11. The chimeric flavivirus vector of claim 1, further comprising a signal sequence at the 3′ end of the capsid gene wherein the signal sequence is from the second flavivirus.

12. The chimeric flavivirus vector of claim 1, wherein the backbone comprises at least one amino acid modification in a non-structural protein selected from the group consisting of NS1 and NS3.

13. The chimeric flavivirus vector of claim 12, wherein the protein is NS1 and the modification is a substitution at an amino acid position corresponding to the proline 315 residue of the Rio Bravo virus NS1 (SEQ ID NO: 1).

14. The chimeric flavivirus vector of claim 13, wherein said proline 315 residue of said NS1 protein is replaced with a serine residue.

15. The chimeric flavivirus vector of claim 12, wherein the protein is NS3 and the modification is a substitution at an amino acid position corresponding to the isoleucine 555 residue of the Rio Bravo virus NS3 (SEQ ID NO: 2).

16. The chimeric flavivirus vector of claim 15, wherein said isoleucine 555 residue of the NS3 protein is replaced with a threonine residue.

17. The chimeric flavivirus vector of claim 1, wherein said chimeric flavivirus vector comprises at least one nucleotide deletion in the 3′ non-coding region (NCR).

18. The chimeric flavivirus vector of claim 17, wherein said at least one nucleotide deletion occurs at a nucleotide corresponding to the thymine at position 10692 of the Rio Bravo virus genome.

19. The chimeric flavivirus vector of claim 1, wherein said chimeric flavivirus vector is within a plasmid comprising at least one cytomegalovirus (CMV) promoter operably linked to the chimeric flavivirus vector.

20. The chimeric flavivirus vector of claim 1, wherein said chimeric flavivirus vector is within a plasmid expressing at least one hepatitis δ virus ribozyme.

21. The chimeric flavivirus vector of claim 1, wherein said chimeric flavivirus vector is within a plasmid comprising at least one SV40 polyadenylation site.

22. The chimeric flavivirus vector of claim 1, wherein the chimeric flavivirus vector comprises one or more intron sequences.

23. The chimeric flavivirus vector of claim 1, wherein the chimeric flavivirus vector comprises an intron immediately after a nucleotide corresponding to position 9742 of the Rio Bravo virus genome

24. The chimeric flavivirus vector of claim 22, wherein the chimeric flavivirus vector comprises an intron at the junction of the envelope (E) and the non-structural gene 1 (NS1).

25. The chimeric flavivirus vector of claim 22, wherein the chimeric flavivirus vector may comprise an intron immediately after a nucleotide corresponding to position 9742 of the Rio Bravo virus genome.

26. A chimeric flavivirus encoded by a chimeric flavivirus vector of claim 1, wherein the chimeric flavivirus is inactivated.

27. The chimeric flavivirus of claim 26, wherein said chimeric flavivirus is inactivated with a method selected from the group consisting of chemical inactivation, high pressure inactivation, ultraviolet radiation, and gamma radiation.

28. The chimeric flavivirus of claim 27, wherein said method of chemical inactivation comprises exposure of the chimeric flavivirus vector to one or more agents selected from the group consisting of β-propiolactone, formalin, aziridines, hydrogen peroxide, organic solvents, and ascorbic acid.

29. A chimeric flavivirus encoded by a chimeric flavivirus vector of claim 1, wherein the chimeric flavivirus is capable of replicating in mammalian cells to at least 10⁸ PFUs/mL.

30. A chimeric flavivirus encoded by a chimeric flavivirus vector of claim 1, wherein the chimeric flavivirus is capable of replicating in insect cells, but not capable of replicating in mammalian cells.

31. A vaccine comprising an inactivated chimeric flavivirus, wherein said inactivated chimeric flavivirus comprises an envelope protein from a first flavivirus with a low level of replication in a cell and 1) a capsid protein from a second flavivirus with a high level of replication in the cell, 2) an RNA genome comprising a sequence encoding one or more non-structural proteins from a second flavivirus with a high level of replication in the cell, or 3) an RNA genome with both 3′ and 5′ termini of the genome from a second flavivirus with a high level of replication in the cell, or a combination thereof, wherein the first flavivirus is different from the second flavivirus.

32. The vaccine of claim 31, wherein the first flavivirus is a dengue virus or a yellow fever virus.

33. The vaccine of claim 31, wherein the first flavivirus is a dengue virus selected from the group consisting of dengue type 1 (DEN1) virus, dengue type 2 (DEN2) virus, dengue type 3 (DEN3) virus, and dengue type 4 (DEN 4) virus.

34. The vaccine of claim 31, wherein said inactivated chimeric flavivirus comprises an envelope protein and a membrane protein from the first flavivirus.

35. The vaccine of claim 31, wherein the second flavivirus is selected from the group consisting of the Rio Bravo taxonomic group, the Uganda S taxonomic group, the mosquito-associated flaviviruses, and the West Nile virus.

36. The vaccine of claim 31, wherein the backbone is from a flavivirus adapted to a cell substrate.

37. The vaccine of claim 31, wherein the backbone is from a flavivirus adapted to a cell substrate such that an increase in yield of the flavivirus is at least about 2.5-fold as compared to the yield of the flavivirus before adaptation.

38. The vaccine of claim 31, further comprising an adjuvant.

39. The vaccine of claim 38, wherein said adjuvant is selected from the group consisting of aluminum hydroxide, MF59, saponin, lipid A, iscomatrix, and immunostimulatory oligonucleotides.

40. A vaccine comprising a chimeric flavivirus encoded by the chimeric flavivirus vector of claim 1.

41. The vaccine of claim 40, wherein the second flavivirus is a mosquito-associated flavivirus and wherein the chimeric flavivirus is not chemically inactivated and the vaccine does not include an exogenous adjuvant.

42. The vaccine of claim 40, wherein the second flavivirus is a mosquito-associated flavivirus, wherein the chimeric flavivirus is capable of replicating in insect cells, but not capable of replicating in human cells, and wherein the vaccine does not include an exogenous adjuvant.

43. A vaccine comprising a mixture of chimeric flaviviruses encoded by a first, second, third or fourth chimeric flavivirus vector of claim 1, wherein the envelope protein in the first, second, third and fourth chimeric flavivirus is from dengue type 1 (DEN1) virus, dengue type 2 (DEN2) virus, dengue type 3 (DEN3) virus, and dengue type 4 (DEN 4) virus, respectively.

44. The vaccine of claim 31, comprising a mixture of a first, second, third and fourth inactivated chimeric flavivirus, wherein the envelope protein in the first, second, third and fourth inactivated chimeric flavivirus is from dengue type 1 (DEN1) virus, dengue type 2 (DEN2) virus, dengue type 3 (DEN3) virus, and dengue type 4 (DEN 4) virus, respectively.

45. The vaccine of claim 31 or claim 40 in an injectable or mucosal formulation.

46. A method for inducing an immune response to a dengue virus in a subject, said method comprising administering to a subject in need of such treatment a vaccine of claim 31 or claim 40.

47. A method for vaccination against dengue virus comprising administering to a subject in need of such treatment an effective amount of a vaccine of claim 31 or claim 40.

48. A chimeric flavivirus vector encoding a structural protein from a first flavivirus with a low level of replication in a cell and a backbone from a second flavivirus with a high level of replication in the cell, wherein the structural protein is an envelope protein and the backbone comprises the capsid protein, the non-structural proteins and the 3′ and 5′ noncoding termini, and wherein the second flavivirus is a flavivirus does not cause cytopathic effect (CPE) when growing in a cell culture.

49. The chimeric flavivirus vector of claim 48, wherein the second flavivirus allows more than one harvesting of a cell culture fluid from a cell culture when growing in the cell culture.

50. A chimeric flavivirus vector encoding a structural protein from a first flavivirus with a low level of replication in a cell and a backbone from a second flavivirus with a high level of replication in the cell, wherein the structural protein is an envelope protein and the backbone comprises the capsid protein, the non-structural proteins and the 3′ and 5′ noncoding termini, and wherein the second flavivirus is a flavivirus that does not cause an elevation in extracellular DNA release as compared to a mock infection when growing in a cell culture, and as measured at day 3 post-infection.

51. A chimeric flavivirus encoded by a chimeric flavivirus vector of claim 48 or claim 50.

52. A vaccine comprising a chimeric flavivirus of claim 51.

53. A vaccine comprising a chimeric flavivirus vector of any of claim 1, 48, or 50.


SEQUENCE LISTING (2 sequences)

SEQ ID NO: 1 - protein, 353 aa, Rio Bravo virus
DHGCALDTYRKEMRCGDGLMVWNDVNNWHEAYKYYPEDPEIFLASLVEDREKHCGYSPSNHIDLAMWKALEEEINWFLEDQSVDWRIMVGNEEKVFHKTNSSGWERRAGRNSLSWKNWAKSFKLMSWILERKQGNGTYFVGPVGLDECPLANRSWNAFKVSEFGAGILQTRAFIDIQTSNSQFCDQGLIGSGAKDKRIVHGSPWMWMDSYEVNGTLQLQKLEMKYAVECLWPLSHTLGGKGAPESQLILPKKLGGPVSTLNMIKGYATQELGPWKNGALVLERGECPGTKVQILEDCDKRGHSVRSTTQGGVTIPDWCCKHCVLPPLKFTNADGCWYAMEIRPLKTTPGLVYA

SEQ ID NO: 2 - protein, 617 aa, Rio Bravo virus
SIIVFGVGEDPEGEKVSIQDGVYRIMVSSLFGKKQVGVGIWTGNSFHTMWHVTRGAVLNICGRKIVPEWASITEDLISYNGGWRLFTRWDGTEVQVHAYTPDGKVTTTQLLPGSMKVEGGLELGLIPLDFPPGSSGSPIISSDGKIIGLYGNGVLHGDTYCSSIAQTEKKEEDVPQPKTLEGDGWLSKGKITVIDAHPGSGKTHRILPNLVKRAAERKMRTLVLAPTRVVIKEMESALKGMDISFHSSAVSTKTPGSLVDVMCHATFVNRKLIHTPQRNYEVIIMDEAHWTDPSSIAARGYITSQCEMKKCAVVLMTATPPGVDDPWANSNEKIADVEKMIPDEPWKQGYEWITDFEGRTAWFVPSYNAAQGISKALRERGKKVLILTSKTFHDNYPKIQSEKPDFILTTDISEMGANLDVDRVIDPRTTLKPMEKGNIVEVSGEISITPASAAQRRGRVGRVKGKKAEYIYQGTTEMDDSDLICWKEAQMLLDNMDSRQRATCQFYEPEQDKMTEIPGYYRLTEEKRKVFRHLLTQCDFTPWLAWNVAQNTKGIEDRGWLNIGPNQHLVSDENGDPIKYTTPSGRERQLQPVWLDNRMVKEKRDLVSLLEYAQMRR